(19) United States
(12) Patent Application Publication
(10) Pub. No.: US 2004/0197429 A1
(43) Pub. Date: Oct. 7, 2004

(54) Selective COX-2 inhibition from plant extracts

(75) Inventors: Mark G. Obukowicz, Kirkwood, MO (US); Susan L. Hummert, St. Ann, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/817,027

(22) Filed: Apr. 2, 2004

Related U.S. Application Data
(63) Continuation of application No. 10/022,862, filed on Dec. 13, 2001, now abandoned.
(60) Provisional application No. 60/304,207, filed on Dec. 15, 2000.

(51) Int. Cl. A61K 35/78

(52) U.S. Cl. 424/725

(57) ABSTRACT

The present invention is directed toward a method for inhibiting COX-2 in an organism. In particular, the method is preferably directed toward selectively inhibiting COX-2 in an organism. The method comprises administering to the organism an organic extract isolated from a plant wherein such extract inhibits COX-2. A method to purify a composition that exhibits COX-2 inhibition and COX-2 selective inhibition from the organic extract is also provided. In addition, a method for treating and/or preventing COX-2 mediated inflammation or inflammation-associated disorders in an organism is provided.

### CROSS-REFERENCE TO RELATED APPLICATIONS

This application is a continuation of and claims priority to U.S. application Ser. No. 10/022,862, filed Dec. 13, 2001, which claims priority to U.S. Provisional Application Serial No. 60/304,207, filed Dec. 15, 2000, both of which are hereby incorporated herein by reference in their entirety.

### FIELD OF THE INVENTION

The current invention is generally directed toward nutraceuticals that are nonsteroidal anti-inflammatory agents capable of inhibiting cyclooxygenase-2 (COX-2). The present invention relates to a method for inhibition of COX-2, or selective inhibition of COX-2 in an organism by administering to the organism organic extracts isolated from plants wherein such extracts inhibit COX-2 activity. The present invention also relates to purified compositions of the plant organic extracts. In addition, the current invention is directed toward a method for treating and/or preventing COX-2 mediated inflammation or inflammation-associated disorders in an organism.

### BACKGROUND OF THE INVENTION

The prostaglandins are a potent class of biologically active lipid derivatives that play a crucial role in the inflammatory response. The inflammatory response is a localized tissue response to injury or other trauma characterized by pain, heat, redness and swelling. Prostaglandins mediate this response by inhibiting platelet aggregation, increasing vascular permeability, increasing vascular dilation, inducing smooth-muscle contraction and causing the induction of neutrophil chemotaxis. Because of their central role in mediating the inflammatory response, significant efforts have been directed toward elucidating compositions that are capable of inhibiting the biosynthesis of prostaglandins.

Toward that end, prostaglandin biosynthesis has been extensively characterized. Prostaglandins are a group of oxygenated fatty acids that are generally derived from arachidonic acid. The biosynthesis of prostaglandins from arachidonic acid occurs in a three step process that includes 1) hydrolysis of arachidonic acid from phospholipid precursors catalyzed by a phospholipase A₂; 2) cyclooxygenase (“COX”) catalyzed oxygenation of arachidonic acid to prostaglandin G2 (“PGG2”). This COX catalyzed reaction is the first committed and rate limiting step in prostaglandin synthesis; and 3) conversion of prostaglandin G2 to the biologically active end product, prostaglandin, catalyzed by a series of synthases and reductases. Upon their synthesis, prostaglandins exit the cell and act in a hormone-like manner by effecting the target cell via G protein linked membrane receptors.

Inactivation of the COX enzyme is a natural target as a means to inhibit prostaglandin production due to this enzyme's pivotal role in the prostaglandin biosynthetic pathway. It is now known that two gene products possessing COX enzyme activity are expressed, termed COX-1 and COX-2. COX-1 was the first discovered isoform and is constitutively expressed in most tissue types. Because it is constitutively expressed, COX-1 is available to participate in activities requiring a rapid physiological response and causes the production of prostaglandins involved in “house-keeping” functions. For example, COX-1 is responsible for acute production of prostaglandins that regulate vascular homeostasis, maintain gastrointestinal integrity, and maintain kidney function. Thus, COX-1 activity is responsible for the synthesis of prostaglandins required for the maintenance of several cell types.

COX-2, on the other hand, is a recently discovered isoform that is inducibly expressed in response to numerous stimuli such as bacterial lipopolysaccharides, growth factors, cytokines, and phorbol esters. In addition, COX-2 is only expressed in a limited number of cell types including monocytes, macrophages, neutrophils, fibroblasts and endothelial cells. COX-2 expression, but not COX-1 expression, has been shown to increase in rheumatoid synovial tissue. Contrastingly, COX-2 expression is inhibited in response to glucocorticoids and by anti-inflammatory cytokines. Thus, based upon these observations, COX-2 has been shown to be the isoform responsible for mediating the production of prostaglandins that participate in the inflammatory response and inflammatory related disorders. In addition, COX-2 has also been shown to participate in certain cancers, Alzheimer's disease, atherosclerosis, and central nervous system damage resulting from stroke, ischemia and trauma.

Corticosteroids provide one means to reduce effects associated with the inflammatory response. These potent anti-inflammatory agents exert their effect by causing a reduction in the number and activity of immune system cells via various mechanisms. However, prolonged administration of corticosteroids results in drastic side effects that limit the therapeutic value of this class of anti-inflammatory agent.

Nonsteroidal anti-inflammatory drugs (NSAIDs) are also utilized as a means to reduce effects associated with the inflammatory response. The principal pharmaceutical effects of NSAIDs are due to their ability to prevent COX activity resulting in the inhibition of prostaglandin synthesis. Inhibition of prostaglandin synthesis by NSAIDs is anti-pyretic, analgesic, anti-inflammatory, and anti-thrombogenic. However, administration of NSAIDs may also result in severe side effects such as gastrointestinal bleeding, ulcers and incidence of renal problems. NSAIDs also inhibit both COX isoforms to varying degrees. For example, the most common NSAID, aspirin (acetylated derivative of salicylic acid), inhibits prostaglandin biosynthesis by irreversibly inactivating both COX-1 and COX-2 via acetylation of a serine residue located in the arachidonic binding domain. While aspirin inactivates both isoforms, it is 10 to 100 times more effective inactivating COX-1 as opposed to COX-2.

The selective inhibition of COX-2 has been shown to be anti-inflammatory and analgesic without the associated gastric and kidney related toxicity problems. This phenomenon is due to the discovery of NSAIDs that are capable of inhibiting COX-2, which is responsible for the production of prostaglandins that mediate the inflammatory response, without causing the inhibition of COX-1, which is responsible for the production of prostaglandins that maintain both gastrointestinal integrity, and kidney function. Thus, the beneficial effects of NSAIDs are separable from their drastic side effects by the development of COX-2 selective inhibitors.

Toward that end, several drugs that are COX-2 selective inhibitors of prostaglandin synthesis have been developed. The most extensively characterized class of COX-2 selective inhibitor is diarylheterocycles, which include the recently approved drugs celecoxib and rofecoxib. However, other classes include, but are not limited to, acidic sulfonamides, indomethacin analogs, zomepirac analogs, chromene analogs and di-t-butylphenols. For example, U.S. Pat. No. 5,380,738 describes oxazoles which selectively inhibit COX-2, U.S. Pat. No. 5,344,991 describes cyclopentenes which selectively inhibit COX-2, U.S. Pat. No. 5,393,790 describes spiro compounds which selectively inhibit COX-2, WO94/15932 describes thiophene and furan derivatives which selectively inhibit COX-2, and WO95/15316 describes pyrazolyl sulfonamide derivatives which selectively inhibit COX-2.

In order to afford an alternative to drug-based selective COX-2 therapy, it would be highly beneficial to provide nutraceuticals that inhibit COX-2, or even more preferably that selectively inhibit COX-2. A nutraceutical, in this context, is a composition that is a naturally occurring product that can safely be consumed and that exhibits COX-2 inhibitory activity. In particular, it would be highly beneficial to obtain the nutraceutical composition or extract from a plant source due to the ability to derive a large quantity of the nutraceutical from a plant at a relatively affordable cost. These nutraceutical compositions could be utilized in the diet in a preventative manner to maintain a “healthy” physiological state. The nutraceutical compositions could also be used as a means to treat, cure or mitigate an existing inflammatory-related ailment either alone or in combination with another compound as a part of combination therapy.

### SUMMARY OF THE INVENTION

Among the several aspects of the invention therefore, is provided a method for inhibiting the activity of COX-2 in an organism, the method comprising the step of administering to the organism a therapeutically or prophylatically effective amount of an organic extract of a plant, wherein the plant is selected from the order consisting of Agavales, Apocynales, Arales, Asterales, Basidiomycetae, Brassicales, Caryophyllales, Cycadales, Ebenales, Euphorbiales, Fagales, Hydrocharitales, Lamiales, Liliales, Loasales, Malvales, Myrtales, Palmales, Pandanales, Papaverales, Piperales, Polemoniales, Polygalales, Primulales, Ranales, Rhamnales, Rosales, Rubiales, Rutales, Santalales, Sapindales, Scrophulariales, Umbellales, Urticales, and Violales.

Another aspect of the invention is a method for inhibiting the activity of COX-2 in an organism, the method comprising the step of administering to the organism a therapeutically or prophylactically effective amount of an organic extract of a plant, wherein the plant is selected from the order consisting of Agavales, Apocynales, Arales, Asterales, Basidiomycetae, Brassicales, Caryophyllales, Cycadales, Ebenales, Euphorbiales, Fagales, Hydrocharitales, Lamiales, Liliales, Loasales, Malvales, Myrtales, Palmales, Pandanales, Papaverales, Piperales, Polemoniales, Polygalales, Primulales, Ranales, Rhamnales, Rosales, Rubiales, Rutales, Santalales, Sapindales, Scrophulariales, Umbellales, Urticales, and Violales, wherein the organic extract is a purified composition obtained by a method comprising contacting the plant with an organic solvent to remove an extract from the plant wherein the extract inhibits COX-2 activity and then isolating the extract with COX-2 inhibitory activity.

Still another aspect provides a method of treating or preventing COX-2 mediated inflammation or an inflammation-associated disorder in an organism, the method comprising administering to the organism a therapeutically or prophylactically effective amount of the purified composition of an organic plant extract wherein the purified composition is obtained by a method comprising contacting the plant with an organic solvent to remove an extract from the plant wherein the extract inhibits COX-2 activity and then isolating the extract with COX-2 inhibitory activity.

Other features of the present invention will be in part apparent to those skilled in the art and in part pointed out in the detailed description provided below.

### BRIEF DESCRIPTION OF THE DRAWINGS

FIG. 1 depicts COX-2>COX-1 inhibition by a plant extract isolated from Trichilia hirta.

FIG. 2 depicts COX-2>COX-1 inhibition by a plant extract isolated from Capsicum frutescens.

FIG. 3 depicts COX-2>COX-1 inhibition by a plant extract isolated from Tradescantia virginiana.

FIG. 4 depicts COX-2>COX-1 inhibition by a plant extract isolated from Tephrosia purpurea.

FIG. 5 depicts COX-2>COX-1 inhibition by a plant extract isolated from Dracontomelon mangiferum.

FIG. 6 depicts COX-2>COX-1 inhibition by a plant extract isolated from Erythrina rubrinervia.

FIG. 7 depicts COX-2>COX-1 inhibition by a plant extract isolated from Pisonia aculeata.

### ABBREVIATIONS AND DEFINITIONS

To facilitate understanding of the invention, a number of terms and abbreviations as used herein are defined below:

“Purified” means partially purified and/or completely purified. Thus, a “purified composition” may be either partially purified or completely purified.

“Extract” means crude extract, purified extract, and purified composition obtained by purification of the extract.

“COX activity” means the ability of either COX isoform, COX-1 or COX-2, to catalyze the oxygenation reaction of arachidonic acid to PGG2.

“COX inhibitor or COX inhibition” means a composition, agent or extract, purified or otherwise, that prevents either COX isoform, COX-1 or COX-2, from catalyzing the oxygenation reaction of arachidonic acid to PGG2 either in whole or in part.

“Selective inhibition of COX-2” means a composition, agent, or extract, purified or otherwise, which selectively inhibits COX-2 activity over COX-1 activity as determined by the ratio of the percentage of COX-2 inhibition divided by the percentage of COX-1 inhibition, unless otherwise indicated herein.

“IC₅₀” means the concentration (in mol L⁻¹) that reduces a specified response to 50% of its former value. As used herein this value measures the amount of composition, agent or extract (ug extract/ml solvent) causing 50% inhibition of PGE2 production. The IC₅₀ value may be used to determine COX-2 selectivity as specifically set-forth herein.

“Plant or parts thereof” means either the whole plant, or any part of the plant such as an aerial part, fruit, leaf, stem, or root and any combination thereof.

“Order”, as utilized herein, is a taxonomic category of related organisms with a category consisting of a number of similar families.

“Family”, as utilized herein, is a taxonomic category of related organisms ranking below the order and above the genus.

“Species”, as utilized herein, is a fundamental taxonomic category ranking below a genus and consisting of a group of closely related individuals.

COX=the enzyme cyclooxygenase

COX-1=the isoform cyclooxygenase-1

COX-2=the isoform cyclooxygenase-2

NSAIDs=nonsteroidal anti-inflammatory drugs

PGE2=prostaglandin E2

### DESCRIPTION OF THE PREFERRED EMBODIMENT

Applicants have discovered that organic extracts of certain plants or parts therefrom inhibit COX-2 activity. Applicants have also discovered that organic extracts of certain plants or parts therefrom selectively inhibit COX-2 activity. The inhibitory effect is selective because inhibition of COX-2 is greater than inhibition of COX-1. Consequently, organic extracts of such plants or parts therefrom may be used to selectively inhibit the activity of COX-2 in an organism without causing an equivalent inhibition of COX-1 activity. Advantageously, these organic extracts are nutraceuticals that may be safely consumed and provide an alternative to traditional drug-based therapy for COX-2 inhibition.

Accordingly, the extracts of the present invention preferably inhibit COX-2 activity more than COX-1 activity. Preferably, the inhibitory effect of the plant extract on COX-2 is at least about two times greater than its inhibitory effect on COX-1. More preferably, the inhibitory effect on COX-2 is at least about 10 times greater than the inhibitory effect on COX-1. COX enzyme inhibition and selectivity may be determined in accordance with any method generally known to those of ordinary skill in the field, as set forth in more detail below.

In addition to inhibiting COX-2, the organic extracts of the present invention may be isolated from an edible or non-edible plant. In general, plants are classified as non-edible if they are utilized for a purpose other than nourishment and categorized as edible if they are consumed for the purpose of nourishment. For example, medicinal plants are considered non-edible because they are consumed for the purpose of correcting symptoms of illness and are considered too potent to be consumed on a daily basis. Classification of plants as edible versus non-edible, therefore, may be accomplished utilizing references commonly known to those skilled in the art for example, such references include, NAPRALERT; Tyozaburo Tanaka, (Edited by Sasuke Nakoa) Tanaka's Cyclopedia of Edible Plants of the World, Keigaku Publishing Co., Tokyo, Japan, 1976; Stephen Facciola, Cornucopia II: A Source Book of Edible Plants, Kampong Publications, Vista, Calif., 1998; James A. Duke, Database of Phytochemical constituents of GRAS Herbs and Other Economic Plants, CRC Press, Boca Raton, Fla., 1992; and George Macdonald Hocking, Dictionary of Natural Products, Plexus Publishing, Inc., Medford, N.J., 1997. The contents of these references are hereby incorporated in their entirety.

In a particularly preferred embodiment, organic extracts are isolated from plants of the following plant orders: Agavales, Apocynales, Arales, Asterales, Basidiomycetae, Brassicales, Caryophyllales, Cycadales, Ebenales, Euphorbiales, Fagales, Hydrocharitales, Lamiales, Liliales, Loasales, Malvales, Myrtales, Palmales, Pandanales, Papaverales, Piperales, Polemoniales, Polygalales, Primulales, Ranales, Rhamnales, Rosales, Rubiales, Rutales, Santalales, Sapindales, Scrophulariales, Umbellales, Urticales, and Violales. The ability of extracts isolated from plants of these particular orders to inhibit COX-2, selectively inhibit COX-2 and their use is set-forth below in Tables 1-2.

In order to prepare the extracts of the invention, a plant or parts thereof are ground into a fine powder, the resultant powder is extracted with a solvent, and the extraction solvent is removed from the extract. The whole plant may be used or parts of the plant including an aerial part, fruit, leaf, stem, or root and any combination thereof may be used. If desired, the resultant extract may be further purified to yield a purified extract or one or more purified compositions. The grinding step may be accomplished by any commonly known method for grinding a plant substance. For example, the plant or parts thereof may be passed through a grinder to obtain a fine powder.

After the plant or parts thereof have been ground into a fine powder, they are combined with an extraction solvent. The solution is then stirred at a temperature, and for a period of time, that is effective to obtain an extract with the desired inhibitory effects on the activity of COX-2. The solution is preferably not overheated, as this may result in degradation and/or denaturation of proteins in the extract. The solution may be stirred at a temperature between about room temperature (25□ C.) and the boiling point of the extraction solvent. Preferably, the solution is stirred at about room temperature.

The length of time during which the plant powder is exposed to the extraction solvent is not critical. Up to a point, the longer the plant powder is exposed to the extraction solvent, the greater is the amount of extract that may be recovered. Preferably, the solution is stirred for at least 1 minute, more preferably for at least 15 minutes, and most preferably for at least 60 minutes.

The extraction process of the present invention is desirably carried out using an organic solvent or a mixture of organic solvents. organic solvents which may be used in the extraction process of the present invention, include but are not limited to hydrocarbon solvents, ether solvents, chlorinated solvents, acetone, ethyl acetate, butanol, ethanol, methanol, isopropyl alcohol and mixtures thereof. Hydrocarbon solvents which may be used in the present invention include heptane, hexane and pentane. Ether solvents which may be used in the present invention include diethyl ether. Chlorinated solvents which may be used in the present invention include dichloromethane and chloroform. Preferably, the solvent utilized for such extraction is a nonpolar organic solvent, such as dichloromethane or hexane.

The relative amount of solvent used in the extraction process may vary considerably, depending upon the particular solvent employed. Typically, for each 100 grams of plant powder to be extracted, about 500 ml of extraction solvent would be used. The organic solvent may be removed from the extract by any method known in the field of chemistry for removing organic solvents from a desired product, including, for example, rotary evaporation.

It is believed that the inhibitory effect of the plant extract of this invention on the activity of COX-2 is due to the presence of one or more compounds in the extract. Compounds present in the extract which inhibit the activity of COX-2 may be isolated and purified by those of ordinary skill in the art employing methods known in the art. For example, column chromatography and fractional distillation may be used to obtain pure compounds from the plant extract of this invention.

The isolation and purification of particular compounds from the organic plant extracts of this invention may be performed as described in Resch, et al., J. Nat. Prod., 61, 347-350 (1998), the entire contents of which are incorporated by reference herein. The methods disclosed therein may be used to isolate and purify compositions which inhibit COX-2.

The ability of a particular organic extract to inhibit COX-1 or COX-2 is preferably determined by performing COX activity assays utilizing recombinant COX-1 and COX-2. The COX-1 and COX-2 genes may be subcloned from a variety of organisms, however in a preferred embodiment such genes are isolated from human or murine sources, using a variety of procedures known to those skilled in the art and detailed in, for example, Sambrook et al., Molecular Cloning, A Laboratory Manual, 2nd ed., Cold Spring Harbor Laboratory Press, (1989) and Ausabel et al., Short Protocols in Molecular Biology, 3rd. ed., John Wiley & Sons (1995). Additionally, the subcloned portion of the particular COX gene may be inserted into a vector by a variety of methods. In a preferred method, the sequence is inserted into an appropriate restriction endonuclease site(s) in a baculovirus transfer vector pVL1393 utilizing procedures known to those skilled in the art and detailed in, for example, Sambrook et al., Molecular Cloning, A Laboratory Manual, 2nd ed., Cold Spring Harbor Laboratory Press, (1989) and Ausubel et al., Short Protocols in Molecular Biology, 3rd ed., John Wiley & Sons (1995).

The recombinant baculoviruses may be isolated by transfecting an appropriate amount of baculovirus transfer vector DNA into a sufficient quantity of SF9 insect cells along with linearized baculovirus plasmid DNA by the calcium phosphate method or any other method generally know to those skilled in the art. (See M. D. Summers and G. E. Smith, A Manual of Methods for Baculovirus Vectors and Insect Cell Culture Procedures, Texas Agric. Exp. Station Bull. 1555 (1987)). Recombinant viruses may be purified by three rounds of plaque purification and high titer (10⁷-10⁸ pfu/ml) stocks of virus may be prepared.

Preferably, for large scale production, cells may be infected in approximately 10 liter fermentors (0.5×10⁶/ml) with the recombinant virus stock such that the multiplicity of infection is greater than about 0.1. After several hours the cells are centrifuged and the cell pellet is homogenized in an appropriate buffer such as Tris/sucrose (50 mM/25%, pH 8.0). The homogenate may then be centrifuged at an appropriate speed and for an appropriate time (such as 10,000×G for 30 minutes) so as to cause the homogenate to separate into a pellet and supernatant fraction. The resultant supernatant fraction will contain the desired product and may be stored at −80° C. until use.

In order to test organic extracts for COX-2 inhibition and selectivity, standard COX-1 and COX-2 assays may be performed by employing ELISA procedures generally known to those skilled in the art. In such procedures, COX-1 and COX-2 activities are assayed as PGE₂ formed/ug protein/time using ELISA to detect the amount of PGE₂ synthesized from arachindonic acid. PGE₂ formation may be measured using PGE₂ specific antibody. Indomethacin, a non-selective COX-2/COX-1 inhibitor, may be employed as a positive control. The relative ability of various organic extracts to inhibit COX-1 or COX-2 at a particular concentration may be determined by comparing the IC₅₀ value expressed as ug extract/ml solvent resulting in a 50% inhibition of PGE2 production. Selective inhibition of COX-2 may then be determined by the IC₅₀ ratio of COX-1/COX-2. Additionally, any other means to determine COX inhibition known to those generally skilled in the art may be employed.

The extracts of this invention may be used to manage, prevent and/or treat an organism having, or at risk for developing, a condition which is mediated in whole or in part by COX-2. Accordingly, conditions which may be benefitted by inhibition of COX-2 or selective inhibition of COX-2 include, but are not limited to, the treatment of inflammation in an organism, and for treatment of other inflammation-associated disorders, such as, an analgesic in the treatment of pain and headaches, or as an antipyretic for the treatment of fever. For example, extracts of the invention would be useful to treat arthritis, including but not limited to rheumatoid arthritis, spondyloarthopathies, gouty arthritis, osteoarthritis, systemic lupus erythematosus and juvenile arthritis. Such extracts of the invention would be useful in the treatment of asthma, bronchitis, menstrual cramps, tendinitis, bursitis, skin-related conditions such as psoriasis, eczema, burns and dermatitis, and from post-operative inflammation including ophthalmic surgery such as cataract surgery and refractive surgery. Extracts of the invention also would be useful to treat gastrointestinal conditions such as inflammatory bowel disease, Crohn's disease, gastritis, irritable bowel syndrome and ulcerative colitis, and treatment of cancer, including but not limited to the following types of cancer: colon, breast, prostate, bladder, or lung. In yet another preferred use, the extracts of the present invention may also be utilized as chemopreventive agents. Extracts of the invention would be useful in treating inflammation in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like. The extracts would also be useful in the treatment of ophthalmic diseases, such as retinitis, retinopathies, uveitis, ocular photophobia, and of acute injury to the eye tissue. The extracts would also be useful in the treatment of pulmonary inflammation, such as that associated with viral infections and cystic fibrosis. Additionally, the extracts would be beneficial for the treatment of certain central nervous system disorders such as cortical dementias including Alzheimer's disease. The extracts of the invention are useful as anti-inflammatory agents, such as for the treatment of arthritis, with the additional benefit of having significantly less harmful side effects. These extracts would also be beneficial in the treatment of allergic rhinitis, respiratory distress syndrome, endotoxin shock syndrome, atherosclerosis and central nervous system damage resulting from stroke, ischemia and trauma. Additionally, the extracts would be useful in the treatment of pain, including but not limited to postoperative pain, dental pain, muscular pain, and pain resulting from cancer.

The present extracts may also be employed either alone or in combination with other compounds as a part of combination therapy, partially or completely, in place of other conventional anti-inflammatories. For example, such as together with steroids, NSAIDs, 5-lipoxygenase inhibitors, leukotriene receptor antagonists, LTA4 hydrolase inhibitors, and LTC4 synthase inhibitors. Preferably, with combination therapy one will typically combine a drug or drugs and a nutraceutical, such as a plant extract of the current invention, in a manner such that the drug and the nutraceutical have different mechanisms of action, but yet target the same disease. For example, in a typical selection of agents for use in combination therapy to treat arthritis, one could utilize a plant extract of the present invention, which exhibits selective COX-2 inhibition with another agent known to attenuate inflammation associated with arthritis via an independent mechanism.

Those of ordinary skill in the art of preparing pharmaceutical formulations can readily formulate pharmaceutical compositions having plant extracts using known excipients (e.g., saline, glucose, starch, etc.). Similarly, those of ordinary skill in the art of preparing nutritional formulations can readily formulate nutritional compositions having plant extracts. And those of ordinary skill in the art of preparing food or food ingredient formulations can readily formulate food compositions or food ingredient compositions having plant extracts.

In addition, those of ordinary skill in the art can readily determine appropriate dosages that are necessary to achieve the desired therapeutic or prophylactic effect upon oral, parenteral, rectal and other administration forms. Typically, in vivo models (i.e., laboratory mammals) are used to determine the appropriate plasma concentrations necessary to achieve a desired mitigation of inflammation related conditions.

The extracts of the present invention may be employed for the treatment and/or prevention of inflammation-related disorders, as identified above, in a number of organisms. Besides being useful for human treatment, these extracts are also useful for veterinary treatment of companion animals, exotic animals and farm animals, including mammals, rodents, avians, and the like. More preferred animals include horses, dogs, cats, sheep, and pigs.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Sample Preparation

Plants or parts thereof were dried and sliced (“sample”). Samples of organic extracts were prepared from the plants listed in Table 1. The plant order and families that the various samples were prepared from are set-forth in Table 1. In addition, details regarding the use of these some of these plants is set-forth in Table 2. The particular sample was then ground into a fine powder using a coffee grinder. Approximately 100 grams of the resulting powder were added to approximately 500 ml of dichloromethane and stirred at room temperature for about 1 hour. The solvent was then removed by rotary evaporation, leaving several grams of the particular extract.

### Inhibitory Effect of Various Plant Organic Extracts on COX-1 and COX-2 Activity

The particular extracts resulting from the sample preparation procedure detailed above were each evaluated for inhibition of COX-1 and COX-2. The COX-1 and COX-2 inhibition activities were determined in vitro according to the method of Gierse et al., J. Diochem., 305, 479-484 (1995). This method is summarized below.

Preparation of Recombinant COX Baculoviruses

Recombinant COX-1 was prepared by cloning a 2.0 kb fragment containing the coding region of human or murine COX-1 into a BamH1 site of the baculovirus transfer vector pVL1393 (Invitrogen) to generate the baculovirus transfer vectors for COX-1 according to the method of D. R. O'Reilly et al., Baculovirus Expression Vectors: A Laboratory Manual (1992).

Recombinant baculoviruses were then isolated by transfecting 4 ug of baculovirus transfer vector DNA into (2×10⁸) SF9 insect cells along with 200 ug of linearized baculovirus plasmid DNA by the calcium phosphate method. (See M. D. Summers and G. E. Smith, A Manual of Methods for Baculovirus Vectors and Insect Cell Culture Procedures, Texas Agric. Exp. Station Bull. 1555 (1987)). Recombinant viruses were purified by three rounds of plaque purification and high titer (10⁷-10⁸ pfu/ml) stocks of virus were prepared.

For large-scale production, SF9 insect cells were infected in 10 liter fermentors (0.5×10⁶/ml) with the recombinant baculovirus stock such that the multiplicity of infection was 0.1. After 72 hours the cells were centrifuged and the cell pellet was homogenized in Tris/sucrose (50 mM/25%, pH 8.0) containing 1% of 3[(3-cholamidopropyl)dimethylammonio]-1-propanesulfonate (CHAPS). The homogenate was then centrifuged at 10,000×G for 30 minutes, and the resultant supernatant was stored at −80° C. until use.

Recombinant COX-2 was prepared by cloning a 2.0 kb fragment containing the coding region of human or murine COX-2 in accordance with the same method described above for COX-1.

Assay for COX-1 and COX-2 Activities

COX-1 and COX-2 activities were assayed as prostaglandin E2 (PGE2) formed/ug protein/time using ELISA to detect PGE2 synthesized from arachindonic acid. CHAPS-solubilized insect cell membranes containing recombinant COX-1 or COX-2 enzyme were incubated in a potassium phosphate buffer (50 mM, pH 8.0) containing epinephrine, phenol, and heme. Compounds were pre-incubated with the appropriate enzyme for approximately 10-20 minutes. Arachidonic acid (10 uM) was then added to the mixture and the reaction was permitted to occur for ten minutes at room temperature (25° C.).

Any reaction between the arachidonic acid and the enzyme was stopped after ten minutes by transferring 40 ul of reaction mixture into 160 ul ELISA buffer and 25 uM indomethacin. Indomethacin, a non-selective COX-2/COX-1 inhibitor, was utilized as a positive control. The PGE₂ formed was measured by standard ELISA technology utilizing a PGE2 specific antibody (Cayman Chemical).

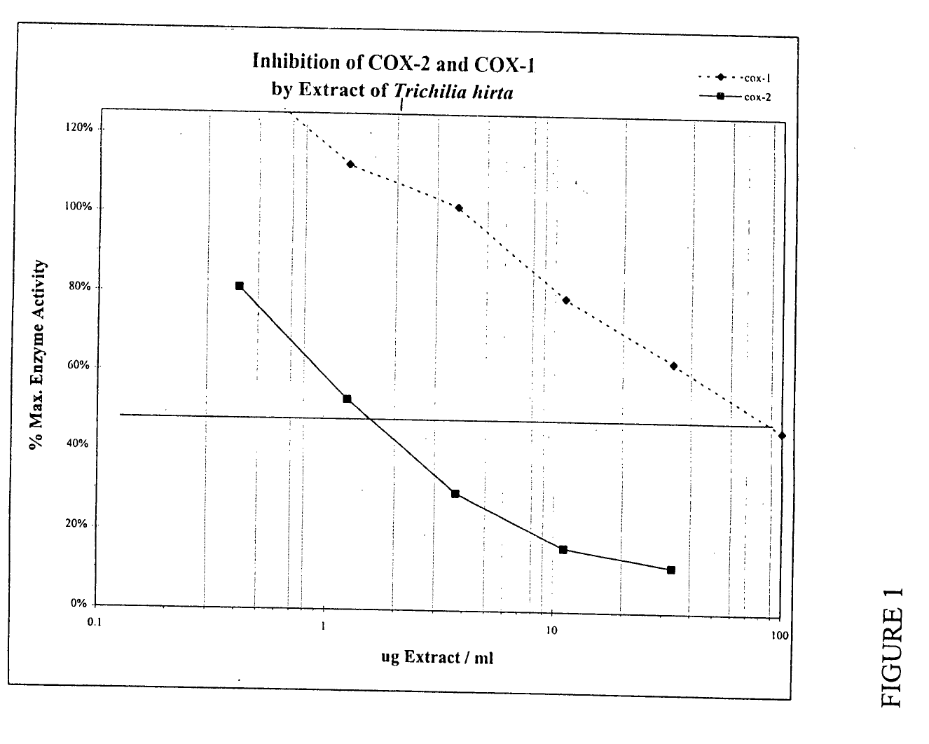

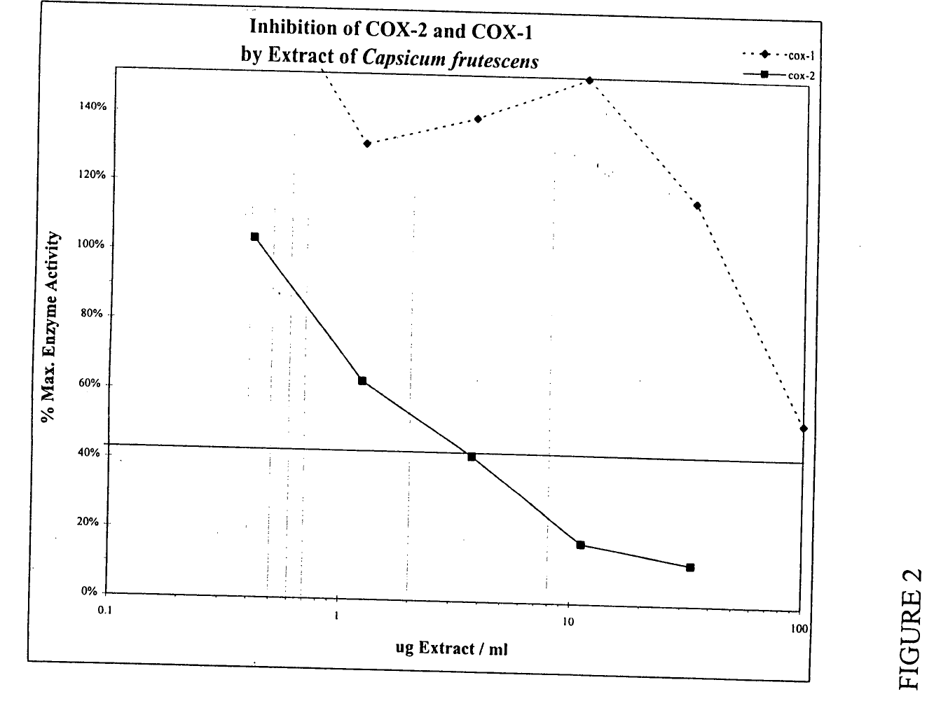

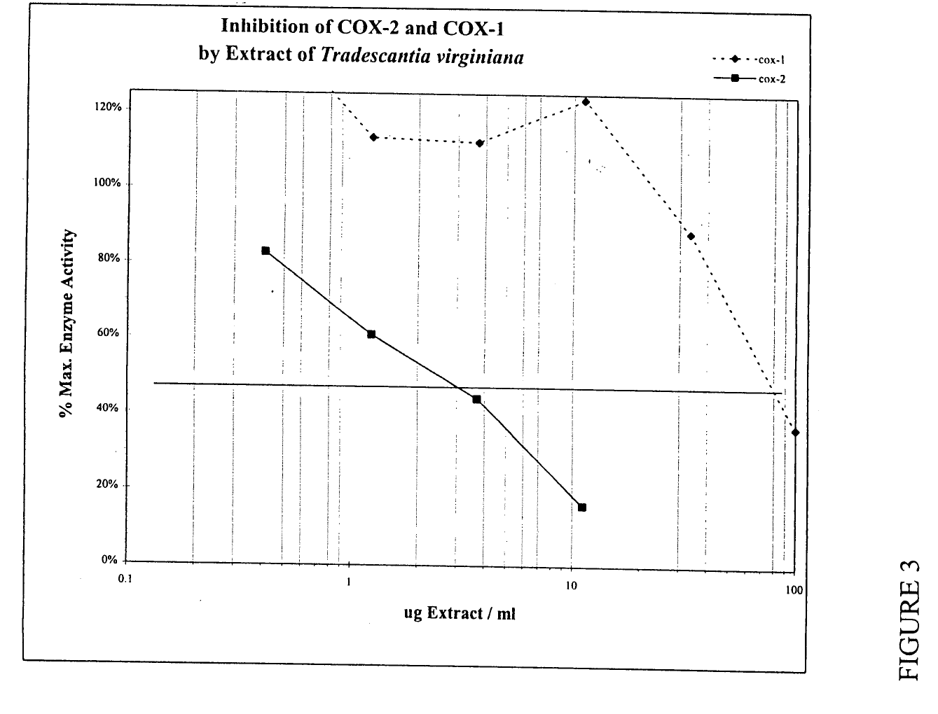

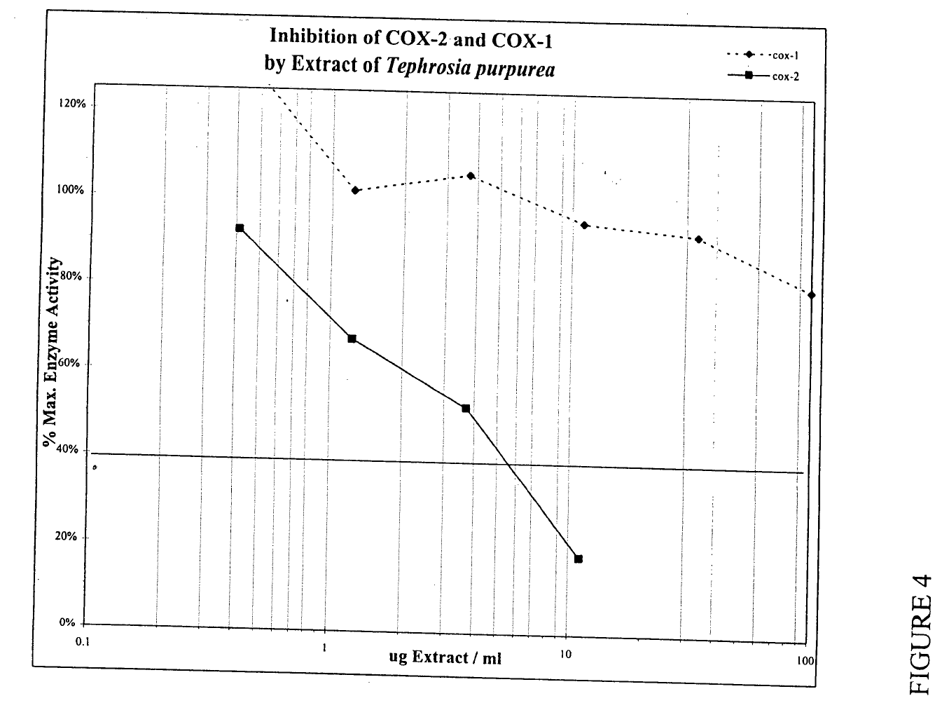

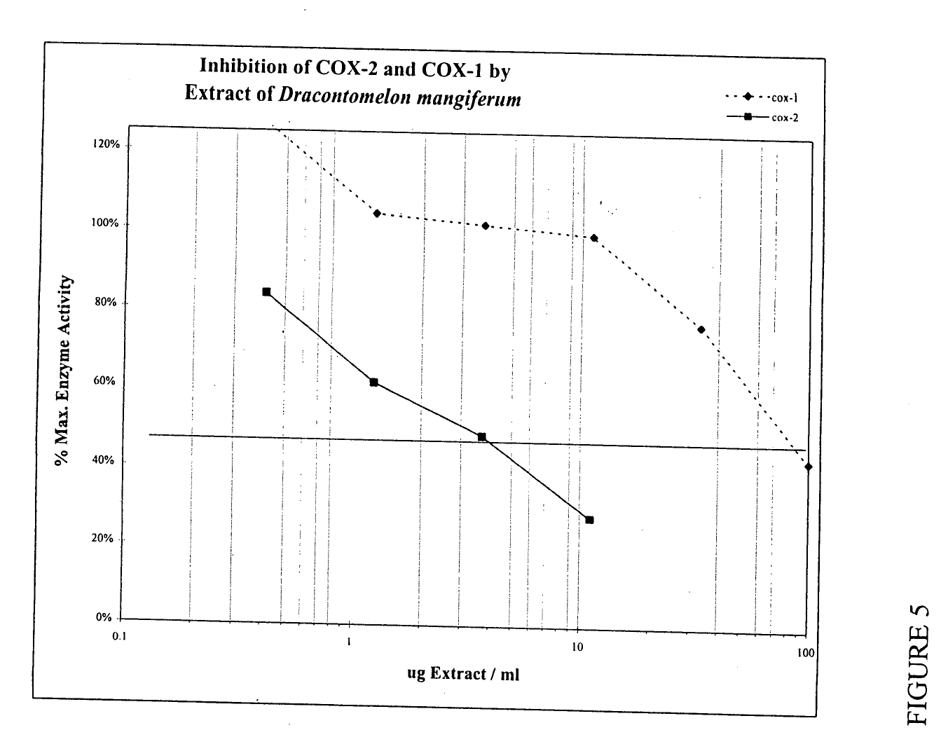

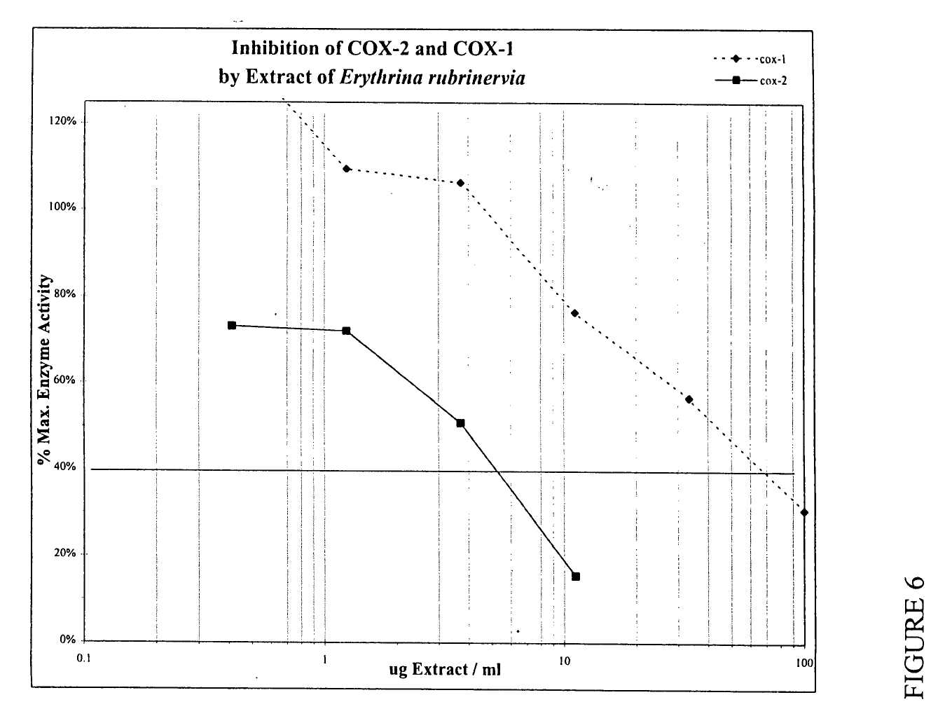

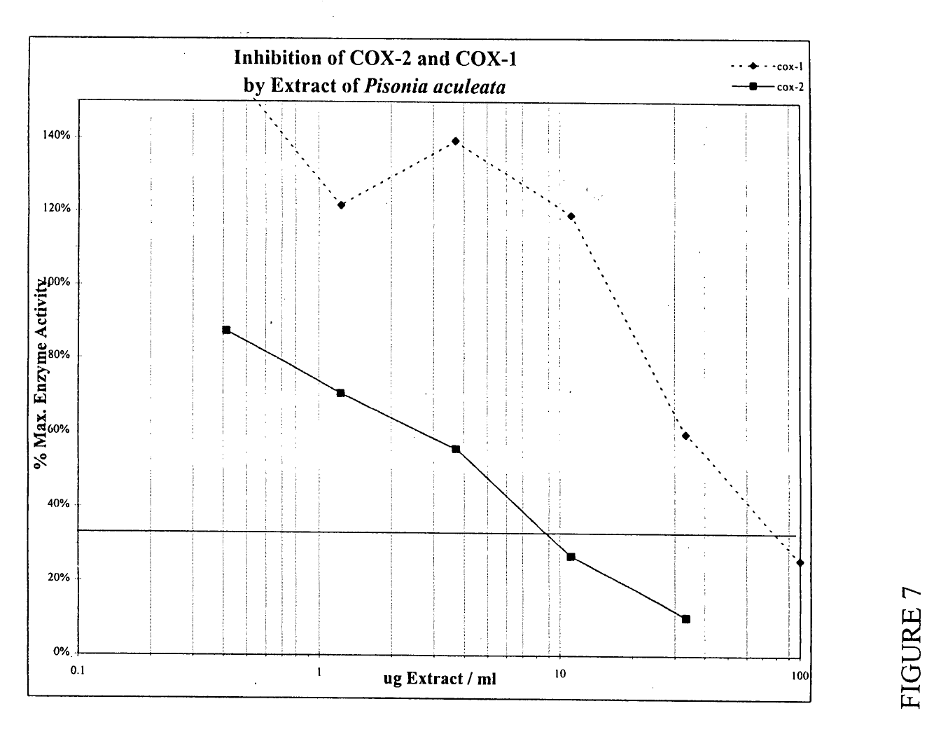

Approximately 200 mg of each extract obtained from the sample preparation procedure set-forth above were each individually dissolved in 2 ml of dimethyl sulfoxide (DMSO) for bioassay testing to determine the COX-1 and COX-2 inhibitory effects of each particular extract. Potency was determined by the IC₅₀ value expressed as ug extract/ml solvent resulting in a 50% inhibition of PGE2 production. Selective inhibition of COX-2 was determined by the IC₅₀ ratio of COX-l/COX-2. The results of these bioassays performed utilizing extract isolated from the plant family indicated are reported in Tables 1 and FIGS. 1-7 delineated below.

Table 1 below sets forth results of screening extracts of plants isolated from the orders, families, genera, and species indicated. A primary screen (indicated as 1° assay in Table 1) was performed in order to determine particular extracts that inhibit COX-2 at a concentration of 10 ug/ml. The extracts were then subjected to a confirmation screen to determine the extent of COX-2 inhibition at three different concentrations (10 ug/ml, 3.3 ug/ml and 1.1 ug/ml). The extracts were then tested for their ability to inhibit COX-1 at a concentration of 10 ug/ml. The percentage of COX inhibition is indicated as a percentage in each column, with a higher percentage indicating a greater degree of COX inhibition. In addition, the IC₅₀ value for COX-1 and COX-2 was also determined for certain extracts as indicated in Table 1. The selectivity for these extracts was then determined by the IC₅₀ ratio of COX-1/COX-2, as set-forth above. The COX-2 selectivity of extracts whose IC₅₀ value was not determined may be calculated by dividing the percentage of COX-1 inhibition (at a concentration of 10 ug/ml) by the percentage of COX-2 inhibition (at a concentration of 10 ug/ml).

TABLE 1
COX-2 Inhibitory Activity from Plant Extracts

| | | | | | | 1* assay | Confirmation assay | | | COX-1 | IC50 | IC50 | |
|---|---|---|---|---|---|---|---|---|---|---|---|---|---|
| | | | | | | COX-2 (% Inhib.) | COX-2 (% Inhib.) | | | (% Inhib.) | (ug/ml) | (ug/ml) | Selectivity |
| Order | Family | Genus | Species | Common name | Part | 10 ug/ml | 10 ug/ml | 3.3 ug/ml | 1.1 ug/ml | 10 ug/ml | COX-2 | COX-1 | COX-2/COX-1 |
| Agavales | Agavaceae | Pleomele | augustifolia | native dracaena | LF | 63% | 73% | 23% | 22% | 40% | *** | *** | *** |
| Apocynales | Apocynaceae | Bleekeria | vitiensis | | | ** | 58% | 34% | −16% | *** | *** | *** | *** |
| Apocynales | Apocynaceae | Strophanthus | hispidus | zwezwe (Africa) | LP | 69% | 72% | 22% | −1% | 6% | *** | *** | *** |
| Apocynales | Asclepiadaceae | Asclepias | asperula | antelope horn | RT | 68% | 70% | 50% | 24% | 1% | *** | *** | *** |
| Arales⁹ | Araceae | Amorphophallus | campanulatus | telinga potato | CO | 70% | 58% | 27% | 2% | −3% | *** | *** | *** |
| Arales⁹ | Araceae | Anthurium | crenatum | | PX | 65% | 65% | 3% | 1% | 23% | *** | *** | *** |
| Arales⁹ | Araceae | Pinellia | ternata | ban xia (China) | | * | 64% | 18% | 2% | *** | *** | *** | *** |
| Arales⁹ | Araceae | Pinellia | ternata | ban xia (China) | | * | 98% | 72% | 38% | 28% | *** | *** | *** |
| Asterales | Asteraceae | Vernonia | sericea | | PX | 77% | 77% | 30% | 19% | 12% | *** | *** | *** |
| Asterales | Asteraceae | Wedelia | reticulata | | PX | 77% | 63% | 37% | 1% | 17% | *** | *** | *** |
| Asterales | Asteraceae | Xanthium | strumarium | arishta (Sanskrit) | FR | 99% | 75% | 62% | 39% | 37% | *** | *** | *** |
| Basidiomycetae | Polyporaceae | Grifola | frondosa | maitake | FB | 67% | 68% | 26% | −2% | 17% | *** | *** | *** |
| Brassicales¹ | Brassicaceae² | Brassica | chinensis | Chinese cabbage | | 50% | 68% | 38% | −5% | *** | *** | *** | *** |
| Brassicales¹ | Brassicaceae² | Brassica | chinensis | Chinese cabbage | | 61% | 22% | 16% | −14% | *** | *** | *** | *** |
| Brassicales¹ | Brassicaceae² | Brassica | oleracea | common cabbage | | * | 41% | 31% | 15% | *** | *** | *** | *** |
| Brassicales¹ | Brassicaceae² | Brassica | oleracea | common cabbage | | * | 74% | 38% | 6% | *** | *** | *** | *** |
| Brassicales¹ | Brassicaceae² | Raphanus | sativus | daikon; semen raphani | | 76% | 81% | 56% | 5% | 25% | *** | *** | *** |
| Brassicales¹ | Brassicaceae² | Raphanus | sativus | daikon; semen raphani | | 71% | 29% | 18% | −10% | *** | *** | *** | *** |
| Brassicales¹ | Brassicaceae² | Raphanus | sativus | daikon; semen raphani | | * | 42% | 34% | 6% | *** | *** | *** | *** |
| Brassicales¹ | Brassicaceae² | Raphanus | sativus | daikon; semen raphani | | * | 54% | 19% | 10% | *** | *** | *** | *** |
| Caryophyllales | Caryophyllaceae | Saponaria | officinalis | soapwort | | * | 30% | 13% | 6% | *** | *** | *** | *** |
| Caryophyllales | Caryophyllaceae | Saponaria | officinalis | soapwort | | * | 42% | −4% | −33% | *** | *** | *** | *** |
| Caryophyllales | Chenopodiaceae | Beta | vulgaris | beet; Swiss chard | RT | 85% | 75% | 48% | 35% | 37% | *** | *** | *** |
| Caryophyllales | Nyctaginaceae | Pisonia | aculeata | cockspur; una de gato | LP | 61% | 97% | 63% | 47% | 6% | 4.5 | 45 | 10 |
| Caryophyllales | Phytolaccaceae | Trichostigma | octandrum | hoop vine | PX | 73% | 82% | 40% | 33% | 43% | *** | *** | *** |
| Caryophyllales | Polygonaceae | Chorizanthe | diffusa | | | 62% | 46% | 31% | −14% | *** | *** | *** | *** |
| Caryophyllales | Polygonaceae | Rumex | hymenosepalus | Indian root; wild rhubarb | | * | 58% | 12% | 5% | *** | *** | *** | *** |
| Caryophyllales | Polygonaceae | Rumex | hymenosepalus | Indian root; wild rhubarb | | * | 83% | 67% | 34% | 36% | *** | *** | *** |
| Cycadales | Cycadaceae | Zamia | debilis | wild sago | | 30% | 66% | 29% | −4% | *** | *** | *** | *** |
| Cycadales | Cycadaceae | Zamia | debilis | wild sago | | 66% | 2% | −11% | −1% | *** | *** | *** | *** |
| Ebenales | Ebenaceae | Diospyros | unidentified | persimmon | LF | 85% | 75% | 53% | 31% | 10% | *** | *** | *** |
| Euphorbiales | Euphorbiaceae | Croton | rigidus | | PX | 69% | 79% | 61% | 21% | 47% | *** | *** | *** |
| Euphorbiales | Euphorbiaceae | Gymnanthes | lucida | | PX | 63% | 79% | 61% | 9% | 53% | *** | *** | *** |
| Euphorbiales | Euphorbiaceae | Macaranga | conifera | | LF | 66% | 64% | 25% | 13% | 36% | *** | *** | *** |
| Euphorbiales | Euphorbiaceae | Macaranga | triloba | mahang serindit (Malaysia) | LF | 62% | 57% | 32% | 48% | 29% | *** | *** | *** |
| Euphorbiales | Euphorbiaceae | Manihot | esculenta | cassava | RB | 71% | 69% | 40% | 21% | 25% | *** | *** | *** |
| Euphorbiales | Euphorbiaceae | Ostodes | paniculata | bijopari | | * | 48% | 11% | −1% | *** | *** | *** | *** |
| Euphorbiales | Euphorbiaceae | Ostodes | paniculata | bijopari | | * | 41% | 15% | −18% | *** | *** | *** | *** |
| Euphorbiales | Euphorbiaceae | Phyllanthus | cuneifolius | | PX | 64% | 62% | 25% | 1% | 46% | *** | *** | *** |
| Euphorbiales | Euphorbiaceae | Ricinodendron | heudelottii | | ST | 73% | 82% | 65% | 32% | 55% | *** | *** | *** |
| Fagales | Fagaceae | Castanopsis | unidentified | | LF | 86% | 64% | 23% | −1% | 41% | *** | *** | *** |
| Fagales | Fagaceae | Castanopsis | unidentified | | LF | 73% | 56% | 15% | 15% | 30% | *** | *** | *** |
| Fagales | Fagaceae | Castanopsis | unidentified | | LF | 66% | 60% | 36% | 4% | 21% | *** | *** | *** |
| Hydrocharilales | Hydrocharitaceae | Elodea | densa | water weed | | 100% | 90% | 78% | 30% | *** | *** | *** | *** |
| Lamiales | Verbenaceae | Callacarpa | cana | | SB | 66% | 71% | 56% | 11% | 45% | *** | *** | *** |
| Lamiales | Verbenaceae | Clerodendron | lecomtei | | LF | 60% | 80% | 54% | 31% | 23% | *** | *** | *** |
| Liliales | Commelinaceae | Tradescantia | virginiana | spiderwort | | ** | 96% | 56% | 25% | 13% | 2.5 | 75 | 30 |
| Liliales | Commelinaceae | Tradescantia | virginiana | spiderwort | | ** | 67% | 48% | −3% | *** | *** | *** | *** |
| Liliates | Liliaceae | Lilium | auratum | goldband lilly | R4 | 73% | 78% | 47% | 31% | 34% | *** | *** | *** |
| Liliales | Liliaceae | Lilium | auratum | goldband lilly | BU | 73% | 70% | 61% | 25% | 40% | *** | *** | *** |
| Liliales | Liliaceae | Lilium | auratum | goldband lilly | | * | 75% | 49% | 30% | 16% | *** | *** | *** |
| Liliales | Liliaceae | Smilax | havanensis | Cuban sarsaparilla | PX | 60% | 80% | −4% | 21% | 15% | *** | *** | *** |
| Loasales | Loasaceae | Mentzelia | aspera | dal pega | PX | 79% | 77% | 46% | 17% | 35% | *** | *** | *** |
| Malvales | Bombaceae⁸ | Quararibea | turbinata | swizzle stick tree | PX | 60% | 70% | 47% | 12% | 19% | *** | *** | *** |
| Malvales | Elaeocarpaceae | Elaeocarpus | bifidus | | | 62% | 85% | 62% | 1% | 12% | *** | *** | *** |
| Malvales | Elaeocarpaceae | Elaeocarpus | bifidus | | | 52% | 37% | 54% | −13% | *** | *** | *** | *** |
| Malvales | Sterculiaceae | Guazuma | ulmifolia | bay cedar | PX | 77% | 71% | 40% | −6% | 17% | *** | *** | *** |
| Malvales | Sterculiaceae | Helicteres | jamaicensis | Jamaican screw tree | PX | 63% | 66% | 32% | 33% | 23% | *** | *** | *** |
| Malvales | Sterculiaceae | Melochia | pyramidata | meloch | PX | 66% | 61% | 14% | −9% | −44% | *** | *** | *** |
| Myrtales | Myrtaceae | Myrcia | splendens | | PX | 61% | 72% | 30% | 23% | 3% | *** | *** | *** |
| Myrtales | Myrtaceae | Syzygium | malaccense | Malay apple | PX | 65% | 62% | 15% | 14% | −7% | *** | *** | *** |
| No order | Cyatheaceae | Cyatheae | unidentified | fern | PE | 73% | 100% | 22% | −13% | 45% | *** | *** | *** |
| No order | Umbilicariaceae | Umbilicaria | proboscidea | umbilicaria lichen | PL | 76% | 81% | 25% | −7% | 61% | *** | *** | *** |
| No order | Boletaceae | Boletus | rubricitrinus | | | 87% | 70% | 59% | 35% | 34% | *** | *** | *** |
| Palmales | Arecacene | Caryota | mitis | sago palm | BK | 61% | 92% | 62% | 40% | 37% | *** | *** | *** |
| Palmales | Arecaceae | Coccothrinax | alta | chestnut; silver palm | PX | 76% | 73% | 35% | 2% | 0% | *** | *** | *** |
| Palmales | Arecaceae | Scheelea | phalerata | scheela palm | LQ | 67% | 76% | 22% | 12% | 42% | *** | *** | *** |
| Pandanales⁹ | Sparganiaceae | Sparganium | ramosum | bur-reed | | * | 58% | 42% | 25% | *** | *** | *** | *** |
| Papaverales | Papaveraceae | Bocconia | frutescens | tree celandine | PX | 71% | 78% | 40% | 39% | 32% | *** | *** | *** |
| Piperales | Chloranthaceae | Hedyosmum | arborescens | | PX | 73% | 54% | 25% | 4% | 7% | *** | *** | *** |
| Piperales | Piperaceae | Peperomia | unidentified | | PL | 95% | 93% | 66% | 28% | 90% | *** | *** | *** |
| Piperales | Piperaceae | Piper | aduncum | pepper | PL | 72% | 83% | 23% | 24% | 34% | *** | *** | *** |
| Polemoniales⁷ | Boraginaceae⁶ | Cordia | laevigata | | PX | 66% | 74% | 42% | 19% | 42% | *** | *** | *** |
| Polemoniales⁷ | Boraginaceae⁶ | Lithospermum | erythrorhizon | red root gromwell | RT | 61% | 70% | 31% | 18% | −21% | *** | *** | *** |
| Polemoniales⁷ | Solanaceae | Capsicum | frutescens | habanero pepper | FR | 60% | 81% | 50% | 12% | 4% | 2.5 | >100 | >40 |
| Polemoniales⁷ | Solanaceae | Capsicum | frutescens | habanero pepper | | * | 59% | 11% | 4% | *** | *** | *** | *** |
| Polemoniales⁷ | Solanaceae | Capsicum | frutescens | habanero pepper | | * | 82% | 64% | 40% | 30% | *** | *** | *** |
| Polemoniales⁷ | Solanaceae | Solanum | acuminatum | | KS | 76% | 81% | 39% | 27% | 48% | *** | *** | *** |
| Polygalales | Polygalaceae | Polygala | penaca | | PX | 71% | 72% | 28% | 22% | 8% | *** | *** | *** |
| Primulales | Myrsinaceae | Myrsine | coriaceae | | PX | 78% | 83% | 58% | 18% | 57% | *** | *** | *** |
| Primulales | Theophrastaceae | Jacquinia | umbellata | | PX | 79% | 79% | 37% | 19% | 30% | *** | *** | *** |
| Primulales | Theophrastaceae | Jacquinia | umbellata | | PX | 75% | 78% | 42% | −2% | 51% | *** | *** | *** |
| Ranales | Lauraceae | Cinnamonum | obtusifolium | cinnamon | LF | 65% | 65% | −22% | −1% | 16% | *** | *** | *** |
| Ranales | Lauraceae | Cinnamonum | parthenoxylon | cinnamon | LF | 79% | 52% | 6% | 6% | −16% | *** | *** | *** |
| Ranales | Ranunculaceae | Paeonia | officinalis | common peony | | 45% | 51% | 15% | −17% | *** | *** | *** | *** |
| Rhamnales | Rhamnaceae | Ziziphus | jujuba | jujube; date tree | SD | 76% | 69% | 61% | 41% | 38% | *** | *** | *** |
| Rhamnales | Rhamnaceae | Ziziphus | jujuba | jujube; date tree | | * | 86% | 72% | 53% | 26% | *** | *** | *** |
| Rhamnales | Rhamnaceae | Ziziphus | jujuba | jujube; date tree | | * | 88% | 74% | 42% | 31% | *** | *** | *** |
| Rosales | Fabaceae | Adenanthera | microsperma | bead tree | LF | 67% | 58% | 29% | 21% | 41% | *** | *** | *** |
| Rosales | Fabaceae | Albizzia | lucida | | LF | 81% | 62% | 26% | 29% | 13% | *** | *** | *** |
| Rosales | Fabaceae | Albizzia | longepedata | | | 68% | −3% | 14% | −14% | *** | *** | *** | *** |
| Rosales | Fabaceae | Cassia | quinquangulata | wampi | | 28% | 57% | 34% | 2% | *** | *** | *** | *** |
| Rosales | Fabaceae | Erythrina | rubrinervia | culantro; gallito | | 68% | 71% | 18% | −32% | *** | *** | *** | *** |
| Rosales | Fabaceae | Erythrina | rubrinervia | culantro; gallito | | 100% | 75% | 36% | −10% | 18% | 4 | 45 | 11 |
| Rosales | Fabaceae | Erythrina | rubrinervia | culantro; gallito | | 50% | 48% | 13% | −43% | *** | *** | *** | *** |
| Rosales | Fabaceae | Erythrina | rubrinervia | culantro; gallito | | 75% | 61% | 42% | −20% | *** | *** | *** | *** |
| Rosales | Fabaceae | Inga | edulis | guavo; ice cream bean | AR | 84% | 82% | 57% | 31% | 34% | *** | *** | *** |
| Rosales | Fabaceae | Milletia | unidentified | | LF | 61% | 81% | 42% | 25% | 46% | *** | *** | *** |
| Rosales | Fabaceae | Tephrosia | purpurea | purple tephrosia | | 60% | 68% | 32% | −9% | *** | *** | *** | *** |
| Rosales | Fabaceae | Tephrosia | purpurea | purple tephrosia | | 98% | 71% | 46% | 8% | 7% | 4 | >100 | >25 |
| Rosales | Rosaceae | Eriobotrya | unidentified | | LF | 80% | 55% | 15% | 10% | 34% | *** | *** | *** |
| Rosales | Saxifragaceae | Mitella | japonica | tyraumeruso | | * | 60% | 28% | 22% | *** | *** | *** | *** |
| Rubiales | Rubiaceae | Berreria | ocymoides | | LF | 79% | 72% | 36% | 10% | 39% | *** | *** | *** |
| Rubiales | Rubiaceae | Genipa | americana | genip | FR | 73% | 63% | 31% | 32% | 28% | *** | *** | *** |
| Rubiales | Rubiaceae | Hamelia | axillaris | yutobanco (Peru) | PX | 75% | 68% | 14% | 2% | 32% | *** | *** | *** |
| Rubiales | Rubiaceae | Hamelia | axillaris | yutobanco (Peru) | PX | 61% | 68% | 38% | 10% | −4% | *** | *** | *** |
| Rubiales | Rubiaceae | Nauclea | orientalis | mau (Burma) | FU | 84% | 57% | 17% | −16% | 44% | *** | *** | *** |
| Rubiales | Rubiaceae | Psychotria | microdon | tapa camino | PX | 74% | 83% | 48% | 11% | 30% | *** | *** | *** |
| Rubiales | Rubiaceae | Psychotria | pubescens | chak k' anan | PX | 74% | 68% | 44% | 31% | 13% | *** | *** | *** |
| Rubiales | Rubiaceae | Psychotria | uliginosa | tres cabezas (Mexico) | PX | 69% | 88% | 70% | 46% | 63% | *** | *** | *** |
| Rubiales | Rubiaceae | Psychotria | unidentified | | BK | 95% | 81% | 66% | 51% | 79% | *** | *** | *** |
| Rutales¹ | Meliaceae | Dysoxylum | excelsum | | LF | 85% | 76% | 27% | 0% | 48% | *** | *** | *** |
| Rutales¹ | Meliaceae | Scindapsus | pictus | | PL | 76% | 70% | 28% | 5% | 23% | *** | *** | *** |
| Rutales¹ | Meliaceae | Trichilia | hirta | broom wood | | 80% | 90% | 61% | 20% | 38% | *** | *** | *** |
| Rutales¹ | Meliaceae | Trichilia | hirta | broom wood | | 98% | 78% | 57% | 3% | 17% | 1.5 | 75 | 50 |
| Rutales¹ | Rutaceae | Clausena | lansium | Chinese wampee | SB | 96% | 85% | 68% | 43% | 44% | *** | *** | *** |
| Rutales¹ | Rutaceae | Clausena | lansium | Chinese wampee | LF | 72% | 70% | 47% | 27% | 40% | *** | *** | *** |
| Rutales¹ | Rutaceae | Zanthoxylum | fagara | wild lime | | * | 40% | 6% | 2% | *** | *** | *** | *** |
| Rutales¹ | Rutaceae | Zanthoxylum | fagara | wild lime | | * | 57% | 24% | 6% | *** | *** | *** | *** |
| Rutales¹ | Rutaceae | Zanthoxylum | piperitum | Japanese pepper | | ** | 64% | 42% | −20% | *** | *** | *** | *** |
| Rutales¹ | Simaroubaceae | Brucea | javanica | kosam seed; java brucea | SD | 66% | 51% | 27% | 16% | 16% | *** | *** | *** |
| Rutales¹ | Simaroubaceae | Picramnia | pentandra | bitter bush | PX | 64% | 55% | 10% | −1% | 14% | *** | *** | *** |
| Santalales | Loranthaceae | Phoradendron | piperoid | pajar (Peru) | PX | 69% | 75% | 43% | 18% | 9% | *** | *** | *** |
| Sapindales¹ | Anacardiaceae | Dracontomelon | dao | argus pheasant tree | FR | 60% | 74% | 53% | 13% | 3% | *** | *** | *** |
| Sapindales¹ | Anacardiaceae | Dracontomelon | mangiferum | sengkuang | | 56% | 79% | 47% | 12% | 3% | 1.8 | 38 | 21 |
| Sapindales¹ | Anacardiaceae | Dracontomelon | unidentified | | | 76% | 86% | 60% | 32% | 2% | 3.5 | 80 | 23 |
| Sapindales¹ | Anacardiaceae | Dracontomelon | unidentified | | | 59% | 40% | −5% | −22% | *** | *** | *** | *** |
| Sapindales¹ | Icacinaceae | Pyrenacantha | staudtii | abere (Nigeria) | | ** | 73% | 50% | 11% | *** | *** | *** | *** |
| Scrophutariales | Bignoniaceae | Macfadyena | unguis-cati | cat's claw | PX | 83% | 69% | 40% | 17% | 29% | *** | *** | *** |
| Scrophulariales | Gesneriaceae | Cyrtandra | grandis | | PL | 74% | 54% | 18% | 10% | 24% | *** | *** | *** |
| Umbellales | Apiaceae⁵ | Apium | graviolens | celery seed | SD | 72% | 68% | 51% | 25% | 30% | *** | *** | *** |
| Umbellales | Araliaceae | Arthophyllum | diversifolium | | LF | 70% | 74% | 36% | −14% | 30% | *** | *** | *** |
| Umbellales | Araliaceae | Arthophyllum | diversifolium | | PE | 69% | 65% | 32% | −15% | 12% | *** | *** | *** |
| Umbellales | Araliaceae | Arthophyllum | diversifolium | | SB | 63% | 49% | −3% | −19% | 8% | *** | *** | *** |
| Umbellales | Araliaceae | VBrassaiopsis | glomerlata | | LF | 69% | 66% | 37% | −8% | 52% | *** | *** | *** |
| Urticales | Moraceae | Dorstenia | contrajerva | contrayerba | PX | 61% | 69% | 23% | 15% | 0% | *** | *** | *** |
| Urticales | Moraceae | Ficus | ribes | fig | LF | 61% | 61% | 34% | 20% | 12% | *** | *** | *** |
| Urticales | Moraceae | Streblus | unidentified | | LF | 88% | 61% | 43% | 27% | 48% | *** | *** | *** |
| Urticales | Ulmaceae | Cellis | unidentified | | LF | 60% | 62% | 19% | 16% | 13% | *** | *** | *** |
| Violales | Flacourtiaceae | Pangium | edule | kluwak; pakem | LF | 96% | 92% | 59% | 45% | 71% | *** | *** | *** |
| Violales | Flacourtiaceae | Pangium | edule | kluwak; pakem | FR | 80% | 75% | 66% | 55% | 82% | *** | *** | *** |
| Violales | Flacourtiaceae | Pangium | edule | kluwak; pakem | BK | 77% | 72% | 28% | 31% | 44% | *** | *** | *** |
| Violales | Flacourtiaceae | Ryparosa | caesia | | TW | 77% | 69% | 31% | −10% | 23% | *** | *** | *** |
| Violales | Flacourtiaceae | Ryparosa | caesia | | LF | 67% | 59% | 20% | −6% | 35% | *** | *** | *** |

* Primary screen performed at three concentrations. Samples were not repeated in a COX-2 confirmation assay.
** No data due to assay error.
*** Not tested.
¹Brassicales also classified as Sapindales or Rutales
²Brassicaceae also classified as Cruciferae
⁵Apiaceae also classified as Umbelliferae
⁶Boraginaceae also classified as Cordiaceae or Ehreliaceae
⁷Polemoniales also classified as Solanales
⁸Bombaceae also classified as Bombacaceae
⁹Pandanales also classified as Arales or Alismatales

The order, family, genus, and species of each plant extract are indicated.

As illustrated by the data in Table 1, the organic extracts isolated from the indicated plant orders inhibit COX-2. In fact, several of the extracts selectively inhibit COX-2 over COX-1 by greater than 10 fold.

Table 2 below provides a description detailing the particular use of some of the plant extracts tested for COX-2 inhibition as set-forth in Table 1. In addition, a comprehensive listing of references known to those generally skilled in the art is provided.

TABLE 2
USES OF PLANT EXTRACTS

| | | Isolate/ | | | |
|---|---|---|---|---|---|
| Scientific Name | Common Name | Chemical ID | Sample ID | Extract # | Reference |
| Adenanthera | bead tree | | | P-01683 | 5 |
| microsperma | | | | | |

Medicinal

| | | |
|---|---|---|
| Albizza lucida | No common | P-01679 |
| | name avail. | |

Seeds are oily and edible.

| | | | |
|---|---|---|---|
| Albizzia | Species | 81259 | 935226 |
| longepedata | not found. | | |

Other species edilble.

| | | | |
|---|---|---|---|
| Amorphophallus | telinga | P-00723 | 3 |
| campanulatus | potato | | |

Leaves and tubers are eaten.

| | | | |
|---|---|---|---|
| Apium graveolens | celery | P-01897 | 1, 2, 3, 4 |

Leaves and leafstalks are used in salad, for flavoring
soups, or as vegetable. The seed is the source of celery, containing
d-limonene, sefinene and sesquiterpene, used in culinary sauces
or for manufacturing celery salt.

| | | | |
|---|---|---|---|
| Asclepias | Antelope | P-00264 | 5 |
| asperula | horns | | |

Medicinal

| | | | |
|---|---|---|---|
| Beta vulgaris | beet or | P-01120 | 1, 2, 3, 4 |
| | Swiss | | |
| | chard | | |

Roots are consumed as vegetable when cooked, in salads.
Leaves are sometimes eaten as potherb.

| | | | | |
|---|---|---|---|---|
| Bleekeria | No common | 81255 | 935185 | 5 |
| vitiensis | name | | | |
| | available. | | | |

Medicinal

| | | | |
|---|---|---|---|
| Bocconia | Ree | P-02163 | 6 |
| frutescens | celandine | | |

Medicinal

| | | |
|---|---|---|
| Boletus | Species | P-01876 |
| rubricitrinus | not found. | |

Fruiting bodies of some species of this mushroom are edible.

| | | | | |
|---|---|---|---|---|
| Brassica | Chinese | 81272 | 935202 | 1, 2, 3, 4 |
| chinensis | cabbage | | | |

Eaten like lettuce.

| | | | | |
|---|---|---|---|---|
| Brassica oleracea | common | 81437 | 936937 | 1, 2, 3 |
| | cabbage | | | |

Eaten raw or cooked.

| | | | |
|---|---|---|---|
| Brucea javanica | kosam | P-00090 | 5 |
| | seed; Java | | |
| | brucea | | |

Medicinal

| | | | |
|---|---|---|---|
| Callicarpa cana | No common | P-01942 | 5 |
| | name | | |

Berries sometimes eaten.

| | | | | |
|---|---|---|---|---|
| Capsicum | habanero | 81442 | 936997 | 1, 2, 3, 4 |
| frutescens | pepper | | | |

Fruits are edible, eaten as vegetable or used as condiment.

| | | | |
|---|---|---|---|
| Caryota mitis | sago palm | P-01601 | 2 |

Buds and seeds are edible.

| | | | | |
|---|---|---|---|---|
| Cassia | wampi | 81274 | 935204 | 5 |
| quinquangulata | | | | |

Medicinal

| | |
|---|---|
| Castanopsis | P-01955 |
| unidentified | |

Fruits of most species edible.

| | |
|---|---|
| Celtis | P-01958 |
| unidentified | |

Species not found, but fruits of some species are edible.

| | | | |
|---|---|---|---|
| Chorizanthe | 81260 | 935227 | 5 |
| diffusa | | | |

Ornamental; not edible

| | |
|---|---|
| Cinnamonum | P-01961 |
| obtusifolium | |

Species not found. Genus of true cinnamons. Edible as condiment.

| | |
|---|---|
| Cinnamonum | P-01964 |
| parthenoxylon | |

Species not found. Genus of true cinnamons. Edible as condiment.

| | | | |
|---|---|---|---|
| Clausena lansium | Chinese | P-01967 | 1, 2, 3 |
| | wampee | | |

The fruit is eaten fresh, preserved, made into jam, pies, or refreshing drinks.
Leaves are put into curries.

| | | |
|---|---|---|
| Clerodendron | P-01969 | 5 |
| lecomtei | | |

Species not found, but others are medicinal.

| | | | |
|---|---|---|---|
| Coccothrinax alta | silver | P-02204 | 5 |
| | palm | | |

Buds and seeds are edible

| | | |
|---|---|---|
| Cordia laevigata | Species | P-02102 |
| | not | |
| | found. | |

The fruits of many species are edible.

| | | |
|---|---|---|
| Croton rigidus | P-02092 | 5 |

Species not found but most other Crotons are poisonous or medicinal.

| | |
|---|---|
| Cyatheae | P-01256 |
| unidentified | |

Other species of this fern used to make a starch.

| | |
|---|---|
| Cyrtandra grandis | P-01741 |

Species not found. Leaves of several other species used as
flavorings or chewed like betel.

| | |
|---|---|
| Diospyros | P-01606 |
| unidentified | |

Genus of persimmons. Fruits of many species edible.

| | | | |
|---|---|---|---|
| Dorstenia | contrayer | P-02213 | 6 |
| contrajerva | ba | | |

Medicinal

| | | | |
|---|---|---|---|
| Dracontomelon dao | argus | P-02250 | 3 |
| | pheasant | | |
| | tree | | |

Fruits are edible, usually mixed with soy sauce in rice.

| | | | | |
|---|---|---|---|---|
| Dracontomelon | sengkuang | 81282 | 935212 | 5 |
| mangiferum | | | | |

Fruits are edible, usually mixed with soy sauce in rice.

| | | |
|---|---|---|
| Dracontomelon | 81283 | 935213 |
| unidentified | | |
| (Draconotomelum) | | |

Fruits of most species edible.

| | | |
|---|---|---|
| Dysoxylum | P-01743 | 5 |
| excelsum | | |

Species not found, but others are medicinal.

| | | | | |
|---|---|---|---|---|
| Elaeocarpus | No common | 81268 | 935235 | 5 |
| bifidus | name. | | | |

Fruits edible.

| | | | | |
|---|---|---|---|---|
| Elodea densa | water | 81278 | 935245 | 5 |
| | weed | | | |
| | genus | | | |

Medicinal

| | |
|---|---|
| Eriobotrya | P-01670 |
| unidentified | |

Species not found. Eriobotrya japonica fruit edible.

| | | | | |
|---|---|---|---|---|
| Erythrina | culantro | 81252 | 935182 | 3 |
| rubrinervia | | | | |

Flowers and flower buds eaten cooked like string beans
in El Salvador and Guatemala. Leaves eaten in soups.

| | | | |
|---|---|---|---|
| Ficus ribes | fig genus | P-01736 | 2 |

Medicinal

| | | | |
|---|---|---|---|
| Genipa americana | genip | P-01810 | 3 |

Fruits are edible when soft and overripe.

| | | | |
|---|---|---|---|
| Grifola frondosa | maitake | P-00001 | 1, 2, 3 |

Fruit bodies are edible.

| | | | |
|---|---|---|---|
| Guazuma ulmifolia | bay cedar | P-02234 | 3 |

Green fruits are eaten raw, cooked, crushed in water to make a beverage, or
used to flavor other foods.

| | | | |
|---|---|---|---|
| Gymnanthes lucida | No common | P-02183 | 5 |
| | name | | |
| | available | | |

Medicinal

| | | | |
|---|---|---|---|
| Hamelia | yutobanco | P-02210 | 5 |
| axillaries | (Peru) | | |

Medicinal

| | | |
|---|---|---|
| Hedyosmum | sago | P-02238 |
| arborescens | palm; | |
| | species | |
| | not found | |

At least on other species (mexicana) has edible fruits and leaves may be used as tea.

| | | | |
|---|---|---|---|
| Helicteres | Jamaican | P-02142 | 5 |
| jamaicensis | screw | | |
| | tree | | |

Medicinal

| | | | |
|---|---|---|---|
| Inga edulis | guavo, | P-02780 | 1, 5 |
| | ice cream | | |
| | bean | | |

Pulp of the fruit is eaten.

| | | | |
|---|---|---|---|
| Jacquinia | Species | P-02137 | 5 |
| umbellata | not | | |
| | found. | | |

Other species are fish poisons or insecticides.

| | | | | |
|---|---|---|---|---|
| Lilium auratum | goldband | 81431 | 936986 | 3 |
| | lily | | | |

Mucilaginous bulb is eaten boiled, sweetened, powdered and added to dumplings.

| | | | |
|---|---|---|---|
| Lithospermum | red root | P-00002 | 5 |
| erythrorhizon | gromwell | | |

Medicinal

| | | | |
|---|---|---|---|
| Macaranga | Species | P-01168 | 5 |
| conifera | not | | |
| | found. | | |

Medicinal

| | | | |
|---|---|---|---|
| Macaranga triloba | Mahang | P-01128 | 5 |
| | serndit | | |
| | (Malaya) | | |

Medicinal

| | | | |
|---|---|---|---|
| Macfadyena | cat's | P-02215 | 5 |
| unguis-cati | claw | | |

Medicinal

| | | | |
|---|---|---|---|
| Manihot esculenta | cassava | P-00204 | 1, 2, 3, 4 |

Young leaves and stems are eaten steamed. Tubers are eaten cooked or fried.
They are ground into flour.

| | | | |
|---|---|---|---|
| Melochia | meloch | P-02127 | 5 |
| pyramidata | | | |

Fruit fermented as a beverage.

| | | | |
|---|---|---|---|
| Mentzelia aspera | dal pega | P-02126 | 5 |

Medicinal

| | | |
|---|---|---|
| Milletia | P02035 | 5 |
| unidentified | | |

Most species used as insecticides, fish poisons and medicinals.

| | | | | |
|---|---|---|---|---|
| Mitella japonica | tyraumeruso | 81439 | 936994 | 5 |

Medicinal

| | | |
|---|---|---|
| Myrcia splendens | P-02236 | 5 |

Medicinal

| | |
|---|---|
| Myrsine coriaceae | P-02159 |

Species not found. Fruit of other species edible.

| | | | |
|---|---|---|---|
| Nauclea | mau | P-01239 | 3 |
| orientalis | (Burmese) | | |

Young leaves and tender tips are steamed and eaten with rice.

| | | | | |
|---|---|---|---|---|
| Ostodes | bijopari | 81445 | 936975 | 5 |
| paniculata | | | | |

Medicinal

| | | | | |
|---|---|---|---|---|
| Paeonia | common | 81266 | 935196 | 3 |
| officinalis | peony | | | |

Hot seeds were ground into a spice in Europe. Mongolians mad a tea from them.
Flowers are eaten as a vegetable or used to scent tea.

| | | | |
|---|---|---|---|
| Pangium edule | pakem | P-02986 | 2 |

Seeds are edible.

| | | |
|---|---|---|
| Peperomia | P-02465 | 5 |
| unidentified | | |

Most species are medicinal.

| | | | |
|---|---|---|---|
| Phoradendron | pajar | P-02205 | 5 |
| piperoid | (Peru) | | |

Medicinal

| | | | |
|---|---|---|---|
| Phyllanthus | Species | P-02144 | 5 |
| cuneifolius | not | | |
| | found. | | |

Medicinal

| | | | |
|---|---|---|---|
| Picramnia | bitter | P-02214 | 5 |
| pentandra | bush | | |

Medicinal

| | | | | |
|---|---|---|---|---|
| Pinellia ternata | ban xia | 81434 | 936989 | 2 |
| | (Chinese) | | | |

Subterranean tubers are edible.

| | | | |
|---|---|---|---|
| Piper aduncum | pepper | P-02466 | 3 |

Peppery fruits used to season foods. Very sweet when black and ripe.
Leaves eaten as potherb.

| | | | |
|---|---|---|---|
| Pisonia aculeate | cockspur; | P-01806 | 5 |
| | una de | | |
| | gato | | |

Medicinal

| | | | |
|---|---|---|---|
| Pleomele | native | P-02692 | 2 |
| angustifolia | dracaena | | |

Young leaves are eaten cooked. Sometimes used to add green color to foodstuff.

| | | | |
|---|---|---|---|
| Psychotria | tapa | P-02099 | 5 |
| microdon | camino | | |

Medicinal

| | | | |
|---|---|---|---|
| Psychotria | chak k' | P-02212 | 5 |
| pubescens | anan | | |

Medicinal

| | | | |
|---|---|---|---|
| Psychotria | tres | P-02077 | 5 |
| uliginosa | cabezas | | |
| | (Mexico) | | |

Medicinal

| | | |
|---|---|---|
| Psychotria | P-01592 | 5 |
| unidentified | | |

Most species are medicinal.

| | | | | |
|---|---|---|---|---|
| Pyrenacantha | abere | 81271 | 935201 | 5 |
| staudtii | (Nigeria) | | | |

Medicinal

| | | | |
|---|---|---|---|
| Quararibea | swizzle | P-02190 | 2, |
| turbinata | stick | | 3, 5 |
| | tree | | |

Twigs used in mixing beverages. Fruit may be edible.

| | | | | |
|---|---|---|---|---|
| Raphanus sativus | daikon, se | 81438 | 936993 | 1, 2, 3, 4 |
| | men | | | |
| | raphani | | | |

Fresh roots are eaten as salad or appetizer, occasionally cooked. Leaves are
eaten as greens. Inflorescences are similarly eaten.

| | | |
|---|---|---|
| Ricinodendron | P-00183 | 2 |
| heudelottii | | |

Probably Ricinodendron heudelotii var. africanum. Seeds are edible.

| | | | | | |
|---|---|---|---|---|---|
| Rumex | Indian | 81450 | 937005 | 937005 | 3 |
| hymenosepalus | root, | | | | |
| | wild | | | | |
| | rhubarb | | | | |

Leafstalks eaten like rhubarb. Leaves eaten after
wash to remove tannins. Seeds are edible.

| | | | |
|---|---|---|---|
| Ryparosa caesia | No common | P-01756 | 2 |
| | name | | |
| | available | | |

Fruit is edible.

| | | | | |
|---|---|---|---|---|
| Saponaria | soapwort | 81451 | 937006 | 3 |
| officinalis | | | | |

An extract of the roots used a an emulsifying agent in foods. The flowers
are occasionally added to salads.

| | | |
|---|---|---|
| Scheelea | scheela | P-02777 |
| phalerata | palm | |

Oil used in cooking

| | | | |
|---|---|---|---|
| Smilax havanensis | Cuban | P-02128 | 5 |
| | sarsaparilla | | |

Medicinal

| | | |
|---|---|---|
| Solanum | P-02461 | 5 |
| acuminatum | | |

Species not found. This is the genus of nightshades, so most are either
medicinal or poisonous.

| | | | | |
|---|---|---|---|---|
| Sparganium | bur-reed | 81433 | 936988 | 2 |
| ramosum | | | | |

Young stems are peeled and boiled down for food.

| | |
|---|---|
| Streblus | P-01665 |
| unidentified | |

Milk from stem of Streblus asper is used to curdle milk. Fruit is edible.

| | | | |
|---|---|---|---|
| Strophanthus | zwezwe | P-00294 | 5 |
| hispidus | (African) | | |

Medicinal

| | | | |
|---|---|---|---|
| Syzygium | Malay | P-02201 | 3 |
| malaccense | apple | | |

Used with seeds to make beverage.

| | | | | |
|---|---|---|---|---|
| Tephrosia | purple | 81267 | 935234 | 1, 2, 3, 4 |
| purpurea | tephrosia | | | |

Seeds used as a substitute for coffee. Roots are used as a flavoring for milk.

| | | | | |
|---|---|---|---|---|
| Tradescantia | spiderwort | 81279 | 935246 | 3 |
| virginiana | | | | |

Very young shoots and leaves eaten in salads. Flowers are an edible garnish.

| | | | | |
|---|---|---|---|---|
| Trichilia hirta | broom | 81264 | 935194 | 5 |
| | wood | | | |

Species not found, but others are medicinal.

| | | |
|---|---|---|
| Trichostigma | hoop vine | P-02162 |
| octandrum | | |

Medicinal

| | | | |
|---|---|---|---|
| Umbilicaria | umbilicaria | P-02749 | 5 |
| proboscidea | lichen | | |

An edible lichen.

| | | | |
|---|---|---|---|
| Veronina sericea | Species | P-02110 | 5 |
| | not | | |
| | found. | | |

Medicinal

| | | | |
|---|---|---|---|
| Wedelia retculata | Species | P-02209 | 5 |
| | not | | |
| | found. | | |

Medicinal

| | | | |
|---|---|---|---|
| Xanthium | arishta | P-01830 | 2 |
| strumarium | (Sanskrit) | | |

Young shoots are eaten cooked, as are young plants. Seeds are ground into
flour and made into noodle. Fruit is sun-dried, roasted and put into
dumplings or cooked with rice.

| | | | | |
|---|---|---|---|---|
| Zamia debilis | wild sago | 81261 | 935228 | 5 |

Tubers are source for starch.

| | | | | |
|---|---|---|---|---|
| Zanthoxylum | wild lime | 81429 | 936959 | 5 |
| fagara | | | | |

Medicinal

| | | | | |
|---|---|---|---|---|
| Zanthoxylum | Japanese | 81247 | 935177 | 1, 2, 3 |
| piperitum | pepper | | | |

Young leaves and fruit are used in dishes; the former being used in Japanese soups,
the latter is cooked into tsukudani. Bark is also employed for seasoning.

| | | | | | |
|---|---|---|---|---|---|
| Ziziphus jujuba | jujube; | 81435 | 936965 | 936965 | 3 |
| | date tree | | | | |

Fruits are edible.

### References

1. NAPRALERT (NATural Products ALERT), which currently contains the extracted information from over 116,000 scientific research articles and books from 1650 A.D. to the present. The NAPRALERT database is housed and maintained by the Program for Collaborative Research in the Pharmaceutical Sciences (PCRPS), within the Department of Medicinal Chemistry and Pharmacognosy, in the College of Pharmacy of the University of Illinois at Chicago, 833 South Wood Street (M/C 877), Chicago, Ill. 60612, U.S.A.

2. Tyozaburo Tanaka, (Edited by Sasuke Nakao) Tanaka's Cyclopedia of Edible Plants of the World, Keigaku Publishing Co., Tokyo, Japan, 1976.

This is a compendium of about 11,000 species of plants, including the essential wild species of the world. This book is considered to be one of the principle references on the world's edible plants.

3. Stephen Facciola, Cornucopia II: A Source Book of Edible Plants, Kampong Publications, Vista, Calif., 1998.

This book records the more than 3,000 species available in the U.S. and abroad.

4. James A. Duke, Database of Phytochemical Constituents of GRAS Herbs and Other Economic Plants, CRC Press, Boca Raton, Fla., 1992.

A database of approximately 1000 plants and 3000 compounds.

5. George Macdonald Hocking, Dictionary of Natural Products, Plexus Publishing, Inc., Medford, N.J., 1997. “Terms in the field of Pharmacognosy relating to natural medicinal and pharmaceutical materials and the plants, animals and minerals from which they are derived.” The work contains over 18,000 entries.

6. Enrique Sanchez-Monge, Flora Agricola: Taxonomia de las Magnoliofitas (Angiospermas) de interes agricola, con excepcion de las de aprovechamiento exclusivamente ornamental o forestall, Ministerio de Agriculture, Pesca y Alimentacion, Madrid, Spain, (date unknown).

An excellent reference work in Spanish with descriptions of plants, common names in many languages and commercial use of agricultural organisms of the world.

7. Anthony R. Torkelson, The Cross Name Index to Medicinal Plants, Volumes !-IV, CRC Press, Boca Raton, Fla., (1998-1999).

8. Umberto Quattrocchi, CRC World Dictionary of Plant Names: Common Names, Scientific Names, Eponyms, Synonyms, and Etymology (Volumes 1-4), CRC Press, Boca Raton, Fla. (2000).

9. W³TROPICOS, a web site providing access to the Missouri Botanical Garden's VAST (VAScular Tropicos) nomenclatural database and associated authority files.

10. Webster's Ninth New Collegiate Dictionary, Merriam-Webster Inc., Springfield, Mass., (1983).

Tables 3-9 further illustrate the ability of certain extracts isolated from the families identified in Table 1 to selectively inhibit COX-2. A total of six different concentrations of the various extracts were tested for their ability to inhibit both COX-1 and COX-2. The IC₅₀ value for COX-1 and COX-2 was also determined and a selectivity ratio was then calculated as set forth above. FIGS. 1-7 are graphs that depict the data shown in Tables 3-9 as indicated.

TABLE 3
Extract isolated from Trichilia hirta

| Amount of | COX-1 Activity | |
|---|---|---|
| Extract | Relative to | COX-2 Activity |
| (ug/ml) | Control | Relative to Control |
| 100 | 46% | Not determined |
| 33.3 | 63% | 11% |
| 11.1 | 79% | 16% |
| 3.70 | 102% | 30% |
| 1.23 | 112% | 53% |
| 0.41 | 135% | 81% |

| IC₅₀ (ug/ml) | IC₅₀ (ug/ml) | C0X-2 |
|---|---|---|
| COX-1 | COX-2 | Selectivity Ratio |
| 75 | 1.5 | 50 |

TABLE 4
Extract isolated from Capsicum frutescens

| Amount of | COX-1 Activity | |
|---|---|---|
| Extract | Relative to | COX-2 Activity |
| (ug/ml) | Control | Relative to Control |
| 100 | 53% | Not determined |
| 33.3 | 116% | 12% |
| 11.1 | 152% | 17% |
| 3.70 | 140% | 42% |
| 1.23 | 132% | 63% |
| 0.41 | 182% | 104% |

| IC₅₀ (ug/ml) | IC₅₀ (ug/ml) | C0X-2 |
|---|---|---|
| COX-1 | COX-2 | Selectivity Ratio |
| >100 | 2.5 | >40 |

TABLE 5
Extract isolated from Tradescantia virginiana

| Amount of | COX-1 Activity | |
|---|---|---|
| Extract | Relative to | COX-2 Activity |
| (ug/ml) | Control | Relative to Control |
| 100 | 37% | Not determined |
| 33.3 | 89% | Not determined |
| 11.1 | 124% | 16% |
| 3.70 | 112% | 44% |
| 1.23 | 113% | 61% |
| 0.41 | 144% | 83% |

| IC₅₀ (ug/ml) | IC₅₀ (ug/ml) | C0X-2 |
|---|---|---|
| COX-1 | COX-2 | Selectivity Ratio |
| 75 | 2.5 | 30 |

TABLE 6
Extract isolated from Tephrosia purpurea

| Amount of | COX-1 Activity | |
|---|---|---|
| Extract | Relative to | COX-2 Activity |
| (ug/ml) | Control | Relative to Control |
| 100 | 80% | Not determined |
| 33.3 | 92% | Not determined |
| 11.1 | 95% | 18% |
| 3.70 | 106% | 52% |
| 1.23 | 102% | 67% |
| 0.41 | 133% | 92% |

| IC₅₀ (ug/ml) | IC₅₀ (ug/ml) | C0X-2 |
|---|---|---|
| COX-1 | COX-2 | Selectivity Ratio |
| >100 | 4 | >25 |

TABLE 7
Extract isolated from Dracontomelon mangiferum

| Amount of | COX-1 Activity | |
|---|---|---|
| Extract | Relative to | COX-2 Activity |
| (ug/ml) | Control | Relative to Control |
| 100 | 25% | Not determined |
| 33.3 | 53% | Not determined |
| 11.1 | 91% | 16% |
| 3.70 | 117% | 39% |
| 1.23 | 114% | 55% |
| 0.41 | 141% | 81% |

| IC₅₀ (ug/ml) | IC₅₀ (ug/ml) | C0X-2 |
|---|---|---|
| COX-1 | COX-2 | Selectivity Ratio |
| 38 | 1.8 | 21 |

TABLE 8
Extract isolated from Erythrina rubrinervia

| Amount of | COX-1 Activity | |
|---|---|---|
| Extract | Relative to | COX-2 Activity |
| (ug/ml) | Control | Relative to Control |
| 100 | 31% | Not determined |
| 33.3 | 57% | Not determined |
| 11.1 | 76% | 16% |
| 3.70 | 106% | 51% |
| 1.23 | 109% | 72% |
| 0.41 | 139% | 73% |

| IC₅₀ (ug/ml) | IC₅₀ (ug/ml) | C0X-2 |
|---|---|---|
| COX-1 | COX-2 | Selectivity Ratio |
| 45 | 4 | 11 |

TABLE 9
Extract isolated from Pisonia aculeata

| Amount of | COX-1 Activity | |
|---|---|---|
| Extract | Relative to | COX-2 Activity |
| (ug/ml) | Control | Relative to Control |
| 100 | 26% | Not determined |
| 33.3 | 60% | 10% |
| 11.1 | 119% | 27% |
| 3.70 | 140% | 56% |
| 1.23 | 122% | 71% |
| 0.41 | 160% | 87% |

| IC₅₀ (ug/ml) | IC₅₀ (ug/ml) | C0X-2 |
|---|---|---|
| COX-1 | COX-2 | Selectivity Ratio |
| 45 | 4.5 | 10 |

As illustrated by these data, the organic extracts isolated from the indicated plants inhibit COX-2. In fact, all of the extracts selectively inhibit COX-2 over COX-1 by greater than or equal to 10-fold. In view of the above, it will be seen that the several objectives of the invention are achieved and other advantageous results attained.

What is claimed is:

1. A method for inhibiting the activity of COX-2 in an organism, the method comprising the step of administering to the organism a composition comprising a therapeutically or prophylatically effective amount of an organic extract of a plant, wherein the plant is selected from the order consisting of Agavales, Apocynales, Arales, Asterales, Basidiomycetae, Brassicales, Caryophyllales, Cycadales, Ebenales, Euphorbiales, Fagales, Hydrocharitales, Lamiales, Liliales, Loasales, Malvales, Myrtales, Palmales, Pandanales, Papaverales, Piperales, Polemoniales, Polygalales, Primulales, Ranales, Rhamnales, Rosales, Rubiales, Rutales, Santalales, Sapindales, Scrophulariales, Umbellales, Urticales, and Violales.

2. The method of claim 1 wherein the inhibitory effect of the extract on COX-2 activity is greater than or equal to about 2 times greater than the inhibitory effect of the extract on COX-1 activity.

3. The method of claim 1 wherein the inhibitory effect of the extract on COX-2 activity is greater than or equal to about 10 times than the inhibitory effect of the extract on COX-1 activity.

4. The method of claim 1 wherein the extract of the Agavales order is from the family Agavaceae.

5. The method of claim 4 wherein the extract of the Agavaceae family is from the Pleomele genus.

6. The method of claim 1 wherein the extract of the Apocynales order is selected from the families consisting of Apocynaceae and Asclepiadaceae.

7. The method of claim 6 wherein the extract of the Apocynaceae family is selected from the genera consisting of Bleekeria and Strophanthus.

8. The method of claim 6 wherein the extract of the Asclepiadaceae family is from the genus Asclepias.

9. The method of claim 1 wherein the extract of the Arales order is from the Araceae family.

10. The method of claim 9 wherein the extract of the Araceae family is selected from the genera consisting of Amorphophallus, Anthurium, and Pinellia.

11. The method of claim 1 wherein the extract of the Asterales order is from the Asteraceae family.

12. The method of claim 11 wherein the extract of the Asteraceae family is selected from the genera consisting of Vernonia, Wedelia, and Xanthium.

13. The method of claim 1 wherein the extract of the Basidiomycetae order is from the Polyporaceae family.

14. The method of claim 13 wherein the extract of the Polyporaceae family is from the genus Grifola.

15. The method of claim 1 wherein the extract of the Brassicales order is from the family Brassicaceae.

16. The method of claim 15 wherein the extract of the Brassicaceae family is from the genera Brassica and Raphanus.

17. The method of claim 1 wherein the extract of the Caryophyllales order is selected from the families consisting of Caryophyllaceae, Chenopodiaceae, Nyctaginaceae, Phytolaccaceae, and Polygonaceae.

18. The method of claim 17 wherein the extract of the Caryophyllaceae family is from the genus Saponaria.

19. The method of claim 17 wherein the extract of the Chenopodiaceae family is from the genus Beta.

20. The method of claim 17 wherein the extract of the Nyctaginaceae family is from the genus Pisonia.

21. The method of claim 17 wherein the extract of the Phytolaccaceae family is from the genus Trichostigma.

22. The method of claim 17 wherein the extract of the Polygonaceae family is from the genera Chorizanthe and Rumex.

23. The method of claim 1 wherein the extract of the Cycadales order is from the family Cycadaceae.

24. The method of claim 23 wherein the extract of the Cycadaceae family is from the genus Zamia.

25. The method of claim 1 wherein the extract of the Ebenales order is from the family Ebenaceae.

26. The method of claim 25 wherein the extract of the Ebenaceae family is from the genus Diospyros.

27. The method of claim 1 wherein the extract of the Euphorbiales order is from the family Euphorbiaceae.

28. The method of claim 27 wherein the extract of the Euphorbiaceae family is selected from the genera consisting of Croton, Gymnanthes, Macaranga, Manihot, Ostodes, Phyllanthus, and Ricinodendron.

29. The method of claim 1 wherein the extract of the Fagales order is from the family Fagaceae.

30. The method of claim 29 wherein the extract of the Fagaceae family is from the genus Castanopsis.

31. The method of claim 1 wherein the extract of the Hydrocharitales order is from the family Hydrocharitaceae.

32. The method of claim 31 wherein the extract of the Hydrocharitaceae family is from the genus Elodea.

33. The method of claim 1 wherein the extract of the Lamiales order is from the family Verbenaceae.

34. The method of claim 33 wherein the extract of the Verbenaceae family is from the genera Callacarpa and Clerodendron.

35. The method of claim 1 wherein the extract of the Liliales order is selected from the families consisting of Commelinaceae and Liliaceae.

36. The method of claim 35 wherein the extract of the Commelinaceae family is from the genus Tradescantia.

37. The method of claim 35 wherein the extract of the Liliaceae family is from the genera consisting of Lilium and Smilax.

38. The method of claim 1 wherein the extract of the Loasales order is from the family Loasaceae.

39. The method of claim 38 wherein the extract of the Loasaceae family is from the genus Mentzelia.

40. The method of claim 1 wherein the extract of the Malvales order is from the families consisting of Bombaceae, Elaeocarpaceae, and Sterculiaceae.

41. The method of claim 40 wherein the extract of the Bombaceae family is from the genus Quararibeae.

42. The method of claim 40 wherein the extract of the Elaeocarpaceae family is from the genus Elaeocarpus.

43. The method of claim 40 wherein the extract of the Sterculiaceae family is from the genera consisting of Guazuma, Helicteres, and Melochia.

44. The method of claim 1 wherein the extract of the Myrtales order is from the Myrtaceae family.

45. The method of claim 44 wherein the extract of the Myrtaceae family is from the genera Myrcia and Syzygium.

46. A method for inhibiting the activity of COX-2 in an organism, the method comprising the step of administering to the organism a composition comprising a therapeutically and prophylatically effective amount of an organic extract of a plant, wherein the plant is from the Boletaceae family and the genus Boletus.

47. A method for inhibiting the activity of COX-2 in an organism, the method comprising the step of administering to the organism a composition comprising a therapeutically and prophylatically effective amount of an organic extract of a plant, wherein the plant is from the Cyatheaceae family and the genus Cyatheae.

48. A method for inhibiting the activity of COX-2 in an organism, the method comprising the step of administering to the organism a composition comprising a therapeutically and prophylatically effective amount of an organic extract of a plant, wherein the plant is from the Umbilicariacae family and the genus Umbilicaria.

49. The method of claim 1 wherein the extract of the Palmales order is from the family Arecaceae.

50. The method of claim 49 wherein the extract of the Arecaceae family is from the genera Caryota, Coccothrinax, and Scheelea.

51. The method of claim 1 wherein the extract of the Pandanales order is from the family Sparganiaceae.

52. The method of claim 51 wherein the extract of the Sparganiaceae family is from the genus Sparganium.

53. The method of claim 1 wherein the extract of the Papaverales order is from the family Papaveraceae.

54. The method of claim 53 wherein the extract of the Papaveraceae family is from the genus Bocconia.

55. The method of claim 1 wherein the extract of the Piperales order is from the families selected from Chloranthaceae, and Piperaceae.

56. The method of claim 55 wherein the extract of the Chloranthaceae family is from the genus Hedyosmum.

57. The method of claim 55 wherein the extract of the Piperaceae family is selected from the genera consisting of Peperomia and Piper.

58. The method of claim 1 wherein the extract of the Polemoniales order is selected from the families consisting of Boraginaceae and Solanaceae.

59. The method of claim 58 wherein the extract of the Boraginaceae family is selected from the genera consisting of Cordia and Lithospermum.

60. The method of claim 58 wherein the extract of the Solanaceae family is selected from the genera consisting of Capsicum and Solanum.

61. The method of claim 1 wherein the extract of the Polygalales order is selected from the family consisting of Polygalaceae.

62. The method of claim 61 wherein the extract of the Polygalaceae family is selected from the genus consisting of Polygala.

63. The method of claim 1 wherein the extract of the Primulales family is selected from the families consisting of Myrsinaceae and Theophrastaceae.

64. The method of claim 63 wherein the extract of the Myrsinaceae family is from the genus Myrsine.

65. The method of claim 63 wherein the extract of the Theophrastaceae family is from the genus Jacquinia.

66. The method of claim 1 wherein the extract of the Ranales order is from the families consisting of Lauraceae and Ranunculaceae.

67. The method of claim 66 wherein the extract of the Lauraceae family is from the genus Cinnamonum.

68. The method of claim 66 wherein the extract of the Ranunculaceae family is from the genus Paeonia.

69. The method of claim 1 wherein the extract of the Rhamnales order is from the family Rhamnaceae.

70. The method of claim 69 wherein the extract of the Rhamnaceae family is from the genus Ziziphus.

71. The method of claim 1 wherein the extract of the Rosales order is selected from the families consisting of Fabaceae, Rosaceae and Saxifragaceae.

72. The method of claim 71 wherein the extract of the Fabaceae family is selected from the genera consisting of Adenanthera, Albizzia, Cassia, Erythrina, Inga, Milletia, and Tephrosia.

73. The method of claim 71 wherein the extract of the Rosaceae family is from the genus Eriobotrya.

74. The method of claim 71 wherein the extract of the Saxifragaceae family is from the genus Mitella.

75. The method of claim 1 wherein the extract of the Rubiales order is from the family Rubiaceae.

76. The method of claim 75 wherein the extract of the Rubiaceae family is selected from the genera consisting of Berreria, Genipa, Hamelia, Nauclea, and Psychotria.

77. The method of claim 1 wherein the extract of the Rutales order is selected from the families consisting of Meliaceae, Rutaceae, and Simaroubaceae.

78. The method of claim 77 wherein the extract of the Meliaceae family is selected from the genera consisting of Dysoxylum, Scindapsus, and Trichilia.

79. The method of claim 77 wherein the extract of the Rutaceae family is selected from the genera consisting of Clausena and Zanthoxylum.

80. The method of claim 77 wherein the extract of the Simaroubaceae family is selected from the genera consisting of Brucea and Picramnia.

81. The method of claim 1 wherein the extract of the Santalales order is from the family Loranthaceae.

82. The method of claim 81 wherein the extract of the Loranthaceae family is from the genus Phoradendron.

83. The method of claim 1 wherein the extract of the Sapindales order is from the families Anacardiaceae and Icacinaceae.

84. The method of claim 83 wherein the extract of the Anacardiaceae family is from the genus Dracontomelon.

85. The method of claim 83 wherein the extract of the Icacinaceae family is from the genus Pyrenacantha.

86. The method of claim 1 wherein the extract of the Scrophulariales order is selected from the families consisting of Bignoniaceae and Gesneriaceae.

87. The method of claim 86 wherein the extract of the Bignoniaceae family is from the genus Macfadyena.

88. The method of claim 86 wherein the extract of the Gesneriaceae family is from the genus Cyrtandra.

89. The method of claim 1 wherein the extract of the Umbellales order is selected from the families consisting of Apiaceae and Araliaceae.

90. The method of claim 89 wherein the extract of the Apiaceae family is from the genus Apium.

91. The method of claim 89 wherein the extract of the Araliaceae family is from the genera Arthophyllum and Brassaiopsis.

92. The method of claim 1 wherein the extract of the Urticales order is selected from the families consisting of Moraceae and Ulmaceae.

93. The method of claim 92 wherein the extract of the Moraceae family is selected from the genera consisting of Dorstenia, Ficus, and Streblus.

94. The method of claim 92 wherein the extract of the Ulmaceae family is from the genus Celtis.

95. The method of claim 1 wherein the extract of the Violales order is from the family Flacourtiaceae.

96. The method of claim 95 wherein the extract of the Flacourtiaceae family is from the genera Pangium and Ryparosa.

97. The method of claim 1 wherein the organic extract is a purified composition obtained by a method comprising:
(a) contacting the plant with an organic solvent to remove an extract from the plant wherein the extract inhibits COX-2 activity; and
(b) isolating the extract with COX-2 inhibitory activity.

98. The method of claim 97 wherein the extract selectively inhibits COX-2 activity.

99. The method of claim 97 wherein step (a) further comprises mixing the plant with the organic solvent and stirring the resulting mixture at a temperature between about 25° C. and the boiling point of said solvent for at least one minute.

100. The method of claim 97 wherein the organic solvent is selected from the group consisting of hydrocarbon solvents, ethers, chlorinated solvents, acetone, ethyl acetate, butanol, ethanol, methanol, isopropyl alcohol and mixtures thereof.

101. The method of claim 97 wherein the organic solvent is non-polar.

102. The method of claim 101 wherein the non-polar organic solvent is dichloromethane or hexane.

103. The method of claim 97 wherein step (b) further comprises separating the solvent from the organic extract by evaporating the solvent.

104. A method of treating or preventing COX-2 mediated inflammation or an inflammation-associated disorder in an organism, the method comprising administering to the organism a composition comprising a therapeutically or prophylactically effective amount of the purified composition according to claim 97.

105. The method of claim 104 wherein the inflammation-associated disorder is arthritis.

106. The method of claim 104 wherein the inflammation-associated disorder is pain.

107. The method of claim 104 wherein the inflammation-associated disorder is fever.

108. The method of claim 104 for use in the treatment or prevention of cancer.

109. The method of claim 108 wherein the cancer is epithelial cell cancer.

110. The method of claim 109 wherein the epithelial cell cancer is colon, breast, prostate, bladder, or lung cancer.

111. The method of claim 104 for use in the treatment or prevention of central nervous system disorders.

112. The method of claim 111 wherein the central nervous system disorder is Alzheimer's Disease.
